SEQUENCE LISTING (14 sequences)

SEQ ID NO: 1 - DNA, 2780 bp, Homo sapiens
  source 1..2780: mol_type = genomic DNA
agagttcacgaggcagccgaggaagaggaggcttgaggcccagggtgggcaccagccagccatggccacagccgagaccgccttgccctccatcagcacactgaccgccctgggccccttcccggacacacaggatgacttcctcaaggtggggcctagaaggtggggtctaggtgggctggctggaatccagggccacagtcacagatcttggggtccagacctgcatcttgacctgaaatcaagagacttaaccaggactgaggtacgctcagtccaggagaggagatctcagcttagtctggcagggggtgaggagggtggtctaggggtttgaggttctaagtgtgatctatttcggtaatagaaaacgaaggtagcctgggcaacatggtgaaaccctatctctacaaaaaataccaaaaacattaggccaggcatgggggcgtgtgcctgtagtcccaggtactccgtaggctgatgcaggaggatcattagagcccaggagattaaggatacagtgagctgcaccactgcactccagcctgggcaaaagagtaagaccctatctcaagaaaaaaaaaaaaaaaaaggaacgagatctaggctcacagacaatcttccagatatcagcgggaaatgatgagtgtgtctgggggacatccaaaatttcggaattaatcttgttttgggagacagggaaggagagggatgttctgggggaaaactaagtcaaggctggcatcctctcccccgtcccctgccagttttccatctccagcagctctgctaccccttcccccatccccgagtgtggttccagatagtggaagtcttatctcctgtctccagccagacctgatcggtttctgtccctggagctggggggggagcggggagaggggcggttagaggggcagtgttggggaagtgggacagacagacaggcaaacaagacccctttccaaagcctctgcgtcagagtgtccagcccgcgatgtccctgggcagggcaccccagtgtccaccgaacctcgagctgcctgctccctcccgcagtggtggcgctccgaagaggcgcaggacatgggcccgggtcctcctgaccccacggagccgcccctccacgtgaagtctgaggaccagcccggggaggaagaggacgatgagaggggcgcggacgccacctgggacctggatctcctcctcaccaacttctcgggcccggagcccggtggcgcgccccagacctgcgctctggcgcccagcgaggcctccggggcgcaatatccgccgccgcccgagactctgggcgcatatgctggcggcccggggctggtggctgggcttttgggttcggaggatcactcgggttgggtgcgccctgccctgcgagcccgggctcccgacgccttcgtgggcccagccctggctccagccccggcccccgagcccaaggcgctggcgctgcaaccggtgtacccggggcccggcgccggctcctcgggtggctacttcccgcggaccgggctttcagtgcctgcggcgtcgggcgccccctacgggctactgtccgggtaccccgcgatgtacccggcgcctcagtaccaagggcacttccagctcttccgcgggctccagggacccgcgcccggtcccgccacgtccccctccttcctgagttgtttgggacccgggacggtgggcactggactcggggggactgcagaggatccaggtgtgatagccgagaccgcgccatccaagcgaggccgacgttcgtgggcgcgcaagaggcaggcagcgcacacgtgcgcgcacccgggttgcggcaagagctacaccaagagctcccacctgaaggcgcatctgcgcacgcacacaggtgagggggcggggccccggacatgagaaagggcgcggcgcccgctgtagttacaggggaagaagggttgcagagggcgggacttggacttggctggcctctgagagtgagtgcctccttaaattttgtgccctaggggcctcactttgttcatcctagtcccagcccaggctgagtaaaggggtgtggccagatgcaggggacccggggacatgactgggcagacagtggcgcttatggcttccttgtcccctaggggagaagccatacgcctgcacgtgggaaggctgcggctggagattcgcgcgctcggacgagctgacccgccactaccggaaacacacggggcagcgccccttccgctgccagctctgcccacgtgctttttcgcgctctgaccacctggccttgcacatgaagcgccacctttgagccctgccctggcacttggactctcctagtgactggggatgggacaagaagcctgtttggtggtctcttcacacggacgcgcgtgacacaatgctgggtggttttcccacgaatggaccctctcctggactcgcgttcccaaagatccacccaaatatcaaacacggacccatagacagccctgggggagcctcttacggaaaatccgacaagccttcagccacagggagccacacagagatgtccaaactgtcgtgcaaacccagtgagacagaccgccaaataaacggactcagtggacactcagaccagctcccagatggccctggacagcaggagagggtgtgggatgaggcttcccagagaccctgggtctagaaagcggctcctgaaggtcccttattgtggctgatattaactgtcaatggttatgggtcctataaaaatgcccctcccagataaa

SEQ ID NO: 2 - DNA, 23 bp, synthetic construct
  source 1..23: mol_type = other DNA
tgtccctggagctggggggggag

SEQ ID NO: 3 - DNA, 23 bp, synthetic construct
  source 1..23: mol_type = other DNA
gggaagtgggacagacagacagg

SEQ ID NO: 4 - protein, 565 aa, synthetic construct
NLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQAHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPAQVVAIASNNGGKQALETVQRLLPVLCQDHGLTPEQVVAIASNGGGKQALETVQRLLPVLCQAHGLTPAQVVAIASNGGGKQALETVQRLLPVLCQDHGLTPDQVVAIASNNGGKQALETVQRLLPVLCQAHGLTPEQVVAIASNNGGKQALE

SEQ ID NO: 5 - protein, 599 aa, synthetic construct
NLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNNGGKQALETVQRLLPVLCQDHGLTPEQVVAIASNGGGKQALETVQRLLPVLCQAHGLTPAQVVAIASNGGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASHDGGKQALETVQRLLPVLCQAHGLTPAQVVAIASHDGGKQALETVQRLLPVLCQAHGLTPAQVVAIASNGGGKQALETVQRLLPVLCQDHGLTPDQVVAIASNNGGKQALETVQRLLPVLCQDHGLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPDQVVAIASNNGGKQALETVQRLLPVLCQAHGLTPDQVVAIASNGGGKQALETVQRLLPVLCQAHGLTPEQVVAIASHDGGKQALE

SEQ ID NO: 6 - protein, 938 aa, synthetic construct
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQAHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPAQVVAIASNNGGKQALETVQRLLPVLCQDHGLTPEQVVAIASNGGGKQALETVQRLLPVLCQAHGLTPAQVVAIASNGGGKQALETVQRLLPVLCQDHGLTPDQVVAIASNNGGKQALETVQRLLPVLCQAHGLTPEQVVAIASNNGGKQALESIVAQLSRPDPALAALLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADAMQSYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 7 - protein, 972 aa, synthetic construct
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNNGGKQALETVQRLLPVLCQDHGLTPEQVVAIASNGGGKQALETVQRLLPVLCQAHGLTPAQVVAIASNGGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASHDGGKQALETVQRLLPVLCQAHGLTPAQVVAIASHDGGKQALETVQRLLPVLCQAHGLTPAQVVAIASNGGGKQALETVQRLLPVLCQDHGLTPDQVVAIASNNGGKQALETVQRLLPVLCQDHGLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPDQVVAIASNNGGKQALETVQRLLPVLCQAHGLTPDQVVAIASNGGGKQALETVQRLLPVLCQAHGLTPEQVVAIASHDGGKQALESIVAQLSRPDPALAALLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADAMQSYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 8 - RNA, 23 nt, synthetic construct
  source 1..23: mol_type = other RNA
tgtccctggagctggggggggag

SEQ ID NO: 9 - RNA, 23 nt, synthetic construct
  source 1..23: mol_type = other RNA
gggaagtgggacagacagacagg

SEQ ID NO: 10 - protein, 21 aa, synthetic construct
MVYPYDVPDYAELPPKKKRKV

SEQ ID NO: 11 - protein, 134 aa, synthetic construct
DLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPL

SEQ ID NO: 12 - protein, 212 aa, synthetic construct
IVAQLSRPDPALAALLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADAMQSYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 13 - DNA, 17 bp, Homo sapiens
  source 1..17: mol_type = genomic DNA
tagaggggcagtgttgg

SEQ ID NO: 14 - DNA, 18 bp, Homo sapiens
  source 1..18: mol_type = genomic DNA
gacagacaggcaaacaag

(19) United States
(12) Patent Application Publication
(10) Pub. No.: US 2024/0335561 A1
(43) Pub. Date: Oct. 10, 2024

(54) A METHOD FOR IN VIVO GENE THERAPY TO CURE SCD WITHOUT MYELOABLATIVE TOXICITY

(71) Applicant: ICAHN SCHOOL OF MEDICINE AT MOUNT SINAI, New York, NY (US)

(72) Inventors: Jeffrey A. Glassberg, New York, NY (US); James J. Bieker, New York, NY (US)

(21) Appl. No.: 18/578,960

(22) PCT Filed: Jul. 22, 2022
(86) PCT No.: PCT/US2022/074043

§ 371 Date: Jan. 12, 2024

Related U.S. Application Data
(60) Provisional application No. 63/281,126, filed on Nov. 19, 2021.
(60) Provisional application No. 63/224,937, filed on Jul. 23, 2021.

(51) Int. Cl. A61K 48/00; A61K 31/395; A61K 38/19; A61K 38/42; A61P 7/06; C12N 9/22; C12N 15/11

(52) U.S. Cl. CPC A61K 48/005; A61K 31/395; A61K 38/193; A61K 38/42; A61K 48/0075; A61P 7/06; C12N 9/22; C12N 15/111; C12N 2310/20

(57) ABSTRACT

Disclosures herein are directed to compositions and methods for increasing gamma globinexpression in a cell or subject by suppressing expression of Klf1. Also described are methods of treating or ameliorating-hemoglobinopathies. The compositions are administered to a subject as a therapeutic amount of a gene editing composition, including an RNA-guided endonuclease and one or more gRNA or sgRNA, or one or more transcription activator-like effector nucleases (TALENs), that targets an intron of a Klf1 gene locus in at least one cell in the subject to effect a deletion in intron, thereby reducing expression of Klf1.

### CROSS-REFERENCE TO RELATED APPLICATION

This application claims the benefit of U.S. Provisional Application No. 63/281,126 filed Nov. 29, 2021 and U.S. Provisional Application No. 63/224,937 filed Jul. 23, 2021, the disclosures of which are hereby incorporated by reference in their entirety.

### ACKNOWLEDGEMENT OF GOVERNMENT SUPPORT

This invention was made with government support under Grants Nos. DK046865 and DK048721 awarded by the National Institutes of Health. The government has certain rights in this invention.

### INCORPORATION BY REFERENCE OF SEQUENCE LISTING

This application contains a Sequence listing that has been submitted via Patent Center in a computer readable format and is hereby incorporated by reference in its entirety. The computer readable file, created on Jul. 22, 2022, is named 093698-731290_Sequence Listing.xml and is about 19,000 bytes in size.

### BACKGROUND

### 1. Field

The present inventive concept is directed to gene therapy compositions and methods for treating β-hemoglobinopathies by modifying expression of KLF1.

### 2. Discussion of Related Art

Hemoglobinopathies arise from deficient expression of globin chains (e.g., β-thalassemia or Cooley's anemia) or expression of mutant β-globin (SCD). Together these are the most prevalent anemias caused by single gene mutations in the world. The associated morbidity is alleviated by expression of the dormant g-globin (i.e., fetal) gene, which can follow from fortuitous genetic inheritance (Hereditary Persistence of Fetal Hemoglobin (HPFH)) or by its pharmacological induction with hydroxyurea. These alter the pattern of globin expression control during development, whereby switching of β-like globin expression normally proceeds in an embryonic (e-) to fetal (g-) to adult (β-) globin expression pattern. Critical to the correct establishment of these changes are the controlling transcription factors, including Erythroid Krüppel-like Factor (KLF1/KLF1). KLF1 accomplishes this feat by direct activation of the adult β-globin and indirect repression of the fetal g-globin gene.

New methods of increasing globin expression in cells and/or patients expressing mutant β-globin are needed.

### SUMMARY

The present disclosure is based, at least in part, on the discovery of new methods of increasing gamma globin expression in a cell by selectively reducing expression of KLF1. Accordingly, one aspect of the disclosure is directed to a method of treating a β-hemoglobinopathy in a subject in need thereof, the method comprising: administering to the subject a therapeutic amount of a gene editing composition targeting an intron of a Klf1 gene in at least one cell in the subject to effect a deletion in intron 1, thereby reducing expression of Klf1, wherein reducing expression of Klf1 increases expression of the gamma-globin in the cell and alleviates the β-hemoglobinopathy in the subject.

In various aspects, expression of Klf1 is reduced by about 30-80%. In some aspects, expression of Klf1 is reduced by about 50%

In various aspects of the disclosure, the deletion of intron 1 may be about 25 to 50 bp in size. In some cases, the deletion occurs between nucleotides 891 and 944 according to SEQ ID NO: 1.

In various aspects, the gene editing composition comprises (a) an RNA-guided endonuclease and (b) one or more gRNA or sgRNA or (c) one or more nucleic acids encoding the RNA-guided endonuclease and/or the one or more gRNAs or sgRNAs targeting the intron of the Klf1 gene. In various aspects, the RNA-guided endonuclease may comprise a Cpf1 endonuclease. In further aspects, each gRNAs or sgRNAs may form a ribonucleoprotein complex with the RNA-guided endonuclease. In some aspects, the one or more gRNAs or sgRNAs comprise a spacer sequence corresponding to SEQ ID NO: 2 and/or SEQ ID NO: 3. For example, the gene editing composition may comprise one gRNA or sgRNA comprising a spacer sequence corresponding to SEQ ID NO: 2 or SEQ ID NO: 3. In other aspects, the gene editing composition comprises a first gRNA and a second gRNA, wherein the first gRNA comprises a spacer sequence corresponding to SEQ ID NO: 2 and the second gRNA comprises a spacer seqence corresponding to SEQ ID NO: 3.

In other aspects of the present disclosure, the gene editing composition may comprise two or more transcription activator-like effector nucleases (TALENs) targeting the intron of the Klf1 gene. In various aspects, the one or more TALENs comprise an amino acid sequence comprising SEQ ID NO: 4 and/or SEQ ID NO: 5. In some aspects, the gene editing composition comprise a first TALEN and a second TALEN, wherein the first TALEN comprises an amino acid sequence according to SEQ ID NO: 4 and the second TALEN comprises an amino acid sequence according to SEQ ID NO: 5.

In any of the above or foregoing aspects, the gene editing composition may comprise a nanoparticle, a liposome, a lentivirus and/or an adenovirus.

In any of the methods provided herein, the cell may be a hematopoietic stem cell or other blood progenitor cell. In any of the methods provided herein, the gene editing composition may be administered via intraosseous infusion. In any of the methods provided herein, the gene editing composition may be directly injected into bone marrow of the subject. In some aspects, the methods of treating β-hemoglobinopathy in a subject further comprises administering a stem cell mobilizing agent (e.g., G-CSF, Plerixafor, or a combination thereof). In various aspects, the gene editing composition is injected intravenously into the subject after intravenous administration of the stem cell mobilizing agent. In other aspects, the gene editing composition is injected subcutaneously into the subject after intravenous administration of the stem cell mobilizing agent. In any of these or related aspects, the gene editing composition may be serially injected into the subject until a satisfactory level of gamma expression is achieved (e.g., at least about 20%, at least about 25%, at least about 30%, at least about 35%, at least about 40% total hemoglobin production in the subject, as measured by hemoglobin electrophoresis or hemoglobin fractionation).

In any of the methods of treating β-hemoglobinopathy provided herein, the β-hemoglobinopathy may comprise sickle cell anemia or beta-thalassemia. In various aspects, treating the β-hemoglobinopathy does not comprise or result in myeloablative toxicity. In further aspects, treating the β-hemoglobinopathy comprises reducing anemia, reducing hemolysis, reducing or preventing organ injury, relieving pain, decreasing frequency of acute and/or chronic complications, improving physical function, improving quality of life or any combination thereof.

In another aspect of the present disclosure, a method of increasing expression of a gamma globin in a cell is provided, the method comprising: delivering to the cell: (a) an RNA-guided endonuclease and (b) one or more gRNA or sgRNA, wherein the one or more gRNA or sgRNA target an intron of a Klf1 gene locus to effect a deletion in the intron, thereby reducing expression of Klf1, wherein reducing expression of Klf1 increases expression of the gamma-globin in the cell.

In various aspects, the RNA-guided endonuclease may comprise a Cpf1 endonuclease. In some aspects, the one or more gRNAs or sgRNAs comprise a spacer sequence corresponding to SEQ ID NO: 2 and/or SEQ ID NO: 3. In some aspects, the method comprises delivering one gRNA or sgRNA comprising a spacer sequence corresponding to SEQ ID NO: 2 or SEQ ID NO: 3. In various aspects, the method comprises delivering a first gRNA or sgRNA and a second gRNA or sgRNA, wherein the gRNA or sgRNA comprises a spacer sequence corresponding to SEQ ID NO: 2 and the second gRNA or sgRNA comprises a spacer sequence corresponding to SEQ ID NO: 3. In any of these or related aspects, the method may comprise delivering one or more nucleic acid encoding the RNA-guided endonuclease and/or the one or more gRNA or sgRNA to the cell. In other aspects, the RNA guided endonuclease is delivered as a protein, optionally, as part of a ribonucleoprotein complex with at least one of the gRNAs or sgRNAs.

In another aspect of the present disclosure, a method of increasing expression of a gamma globin in a cell is provided, the method comprising: delivering to the cell one or more transcription activator-like effector nucleases (TALENs) targeting an intron of a Klf1 gene locus to effect a deletion in the intron, thereby reducing expression of Klf1, wherein reducing expression of Klf1 increases expression of the gamma globin in the cell. In various aspects, the one or more TALENs may comprise an amino acid sequence comprising SEQ ID NO: 4 and/or SEQ ID NO: 5. In various aspects, the method comprises delivering a first TALEN and a second TALEN, wherein the first TALEN comprises an amino acid sequence according to SEQ ID NO: 4 and the second TALEN comprises an amino acid sequence according to SEQ ID NO: 5.

In various aspects, the methods provided herein may comprise delivering one or more nucleic acid encoding the one or more TALENs to the cell. In other aspects, the methods may comprise delivering the one or more TALENs as proteins to the cell.

In any of the methods of described herein, expression of Klf1 may be reduced by about 30-80%. For example, expression of Klf1 may be reduced by about 50%. Likewise, in some aspects, the deletion may be about 20 to 30 bp in size. In various aspects, the deletion may occur between nucleotides 891 and 944 according to SEQ ID NO: 1. In any of the methods provided herein, the cell may be a hematopoetic stem cell or other blood progenitor cell. In various aspects, the cell may be in vitro. In various aspects, the cell may be in vivo. In other aspects, the cell may be ex vivo.

Also provided herein are gene editing compositions comprising (a) an RNA-guided endonuclease or nucleic acid encoding the RNA-guided endonuclease, (b) one or more gRNAs or sgRNAs targeting an intron of a Klf1 gene locus or a nucleic acid encoding the one or more gRNAs or sgRNAs and (c) a pharmaceutically acceptable carrier or excipient. In various aspects, the RNA-guided endonuclease may comprise Cpf1. In various aspects, the one or more gRNAs or sgRNAs may each comprise a spacer sequence corresponding to SEQ ID NO: 2 and/or SEQ ID NO: 3. In various aspects, the gene editing composition comprises a first gRNA comprising a spacer sequence corresponding to SEQ ID NO: 2 and a second gRNA comprising a spacer sequence corresponding to SEQ ID NO: 3.

In any of the gene editing compositions herein, each gRNA or sgRNA may form a ribonucleoprotein with at least one RNA endonuclease and the gene editing composition ay comprise the ribonucleoprotein complex. In various aspects, the gene editing composition may comprise one or more nucleic acid encoding the RNA-guided endonuclease and/or the one or more gRNAs or sgRNAs.

Further aspects of the present disclosure are directed to gene editing compositions comprising (a) one or more transcription activator-like effector nucleases (TALENs) targeting an intron of a Klf1 gene locus and (b) a pharmaceutically acceptable carrier or excipient. In various aspects, the at least one TALEN comprises an amino acid sequence comprising SEQ ID NO: 4 or SEQ ID NO: 5. In some aspects, the gene editing composition comprises a first TALEN having an amino acid sequence comprising SEQ ID NO: 4 and a second TALEN having an amino acid sequence comprising SEQ ID NO: 5. In any of the foregoing aspects, the carrier or excipient may comprise a liposome, a nanoparticle, a viral vector or any combination thereof. In some aspects, the gene editing composition may be formulated for intraosseous infusion or bone marrow injection.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein. Embodiments of the present inventive concept are illustrated by way of example in which like reference numerals indicate similar elements and in which:

FIGS. 1A presents representative chromatograms from sequencing assays for the Klf1 intron showing representative mutations in JMML patient samples.

FIG. 1B presents sequence alignment of the intron 1 of the Klf1 gene in various species.

FIG. 2A presents the results from reanalysis of data from a study {Lohmann, 2008 #1509} on the mouse KLF1 promoter. Constructs containing the KLF1 promoter or one that additionally includes intron 1 were stably integrated into mouse ES cells adjacent to a GFP reporter, yielding two stable ES lines (“P-Klf1-GFP” and “P-Klf1-intron-GFP”). Top: Locations of the mapped upstream enhancer, proximal promoter, and intronic enhancer are as indicated. Bottom: ES cells from each line were differentiated to EBs for the indicated number of days, and samples were quantitatively analyzed for expression of endogenous KLF1 to monitor normal expression onset (red; C=control) or exogenous reporter (green) to monitor the effect of intron 1 inclusion.

FIG. 2B presents genome browser data aligned at the human KLF1 genomic region [refs]. Shaded bars show the locations of the enhancer upstream of the gene and of the intron 1 site within the gene (also marked with an asterisk). Top: In vivo GATA1, SMAD1, TAL1, and P300 binding identified from ChIP-seq analyses of erythroid cells. Middle: Onset of chromatin accessibility during erythroid differentiation from human CD34+ cells as monitored by ATAC-seq analyses of sorted cells. Bottom: Comparison of chromatin accessibility (ATAC-seq) of prospectively isolated human hematopoietic populations.

FIG. 2C presents reporter assay of various renilla reporter constructs after transfection into human JK1 cells. Top: Schematic of constructs containing the KLF1 promoter (as in (A)) are shown. The location of the intron 1 point mutant introduced into P-Klf1-intron-Ren is shown (“M”). Bottom: Results of the assay, showing high renilla levels from the promoter alone that are further stimulated by inclusion of the intron (two separate DNA preparations); however these levels are decreased when the point mutant variant is used (three separate preparations). Normalization is to a cotransfected luciferase plasmid, and data is an average of triplicate samples.

FIG. 3 presents reporter assay of various renilla reporter constructs after transfection into human JK1 cells. Top: Schematic of constructs containing the KLF1 promoter are shown. Mutations were introduced in two upstream promoter regions and levels of renilla expression measured (bottom).

FIG. 4A presents a schematic of the location of the two TALEN arms (blue) in the context of the KLF1 gene (SEQ ID NO: 1) and the intron 1 mutation site (red).

FIG. 4B presents plots of KLF1 RNA expression and western blot assessment of KLF1 and HSP90 protein in KU812 cell clones (FIG. 5B) compared to parental cells.

FIG. 4C presents a plot of RNA expression of epsilon globin in KU812 clones [note scale change].

FIG. 4D presents a plot of relative occupancy as evaluated by ChIP analysis of specific TEL/ETV6 binding in WT or mutant (clone 2, “Mut”) KU812 cells. Left, positive control targets known to bind ETV6 show signals in all cases (HHEX, GATA2, LMO2) in WT or mutant cells. Right, ETV6 occupancy at the KLF1 intron 1 site, monitored by two different primer pairs, show a positive signal only WT cells.

FIG. 5A presents a diagram of a dual RFP/GFP reporter system used herein and a representative flow cytometry plot showing how the system was used to isolate human KU812 cells successfully transfected with TALENs targeting a site within the Klf1 gene.

FIG. 5B presents a sequence alignment of a portion of the Klf1 gene intron 1 for parental cell lines and two clones following transfection with TALENs targeting the Klf1 gene.

FIG. 6 presents results from a UniPROBE factor interaction prediction analysis showing binding sites of different transcription factors (left) along the region of the Klf1 gene disrupted by intronic mutations.

FIG. 7A depicts NGS genomic analysis (left) and KLF1 expression levels (right) of CD34+ cells edited with TALEN arms targeting sequences in yellow (upper left) and designed to excise the location of the JMML point mutation (illustrated in sequence in red).

FIG. 7B depicts NGS genomic analysis (left) and KLF1 expression levels (right) of CD34+ cells edited with a CRISPR/Cpf1 gene editing system using one gRNA targeting the sequence in yellow (upper left).

FIG. 7C depicts NGS genomic analysis (left) and KLF1 expression levels (right) of CD34+ edited with a CRISPR/Cpf1 gene editing system using two gRNAs targeting the indicated sequences in yellow (upper left).

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part, on the discovery that Erythroid Krüppel-like Factor (KLF1) expression can be reduced to increase gamma globinexpression in blood cell precursors. The methods provided herein are advantageous because KLF1 is only expressed in blood cell precursors, such as hematopoetic stem cells. Accordingly, methods to reduce its expression are limited to those cells in the blood cell lineage—reducing harmful off-target effects. Further, the methods provided herein allow for targeted reduction, but not elimination, of KLF1 expression by targeting a newly discovered intronic enhancer of the Klf1 gene. These methods advantageously allow for targeted replenishment of globin expression in patient blood cells. Thus, this approach, unlike other methods, can be applied within the human body without the need for toxic myeloablative therapy.

### I. TERMINOLOGY

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “nucleic acid” or “polynucleotide” refers to deoxyribonucleic acids (DNA) or ribonucleic acids (RNA) and polymers thereof in either single-or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, v variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. GENE EDITING COMPOSITIONS

The present disclosure provides for gene-editing compositions for preventing, ameliorating or treating one or more β-hemoglobinopathies. In some aspects, the gene-editing compositions comprise one or more components that together delete (or excise) a portion of an intron of the Klf1 gene (e.g., a portion in intron 1 of the Klf1 gene) in a cell. In various aspects, the portion of the Klf1 gene intron comprises an intronic enhancer, which when excised, leads to reduction (but not elimination) of KLF1 expression. For ease of reference, the full Klf1 gene is provided herein as SEQ ID NO: 1. Intron 1 is located from nucleotide 149 to nucleotide 1059, according to SEQ ID NO: 1 and the targeted intronic enhancer comprises nucleotide 932. In various aspects, the gene-editing compositions provided herein delete about 10 to 50 base pairs, about 20 to 40 base pairs, or about 20 to 30 base pairs around nucleotide 932. In some examples, the deletion may occur between nucleotides 800 and 1000, between nucleotides 850 and 960, or between nucleotides 860 and 960. In some examples, the deletion may occur between nucleotides 866 and 952. In some examples, the deletion may occur between nucleotides 891 and 944. In various aspects, excision of the intronic enhancer results in about 30-80% drop in KLF1 expression (e.g., about a 30%, about a 40%, about a 50%, about a 60%, about a 70%, or about an 80% drop). In some aspects, excision of the intronic enhancer results in about 50% drop in KLF1 expression.

In various aspects, therefore, the gene-editing composition may comprise an endonuclease capable of cleaving the gene at one or more targeted locations such that a portion of the Klf1 gene (e.g., an intronic enhancer) at or between these targeted locations is excised. In various aspects, the gene-editing composition comprises a Clustered Regularly Interspaced Short Palindromic Repeats (CRISPR) associated (Cas) system comprising a CRISPR-associated (Cas) endonuclease and one or more gRNAs. In other aspects, the gene-editing composition comprises one or more of an alternative gene targeting endonuclease (e.g., a transcription activator-like effector nuclease or TALEN(s)). Each gene editing system is described in more detail below. In further embodiments, the compositions herein may further comprise vectors, nucleic acids, delivering vesicles, carriers and/or excipients to facilitate delivery of the gene-editing components to a cell in vitro, ex vivo, or in vivo. In some embodiments, compositions herein can be formulated to form one or more pharmaceutical compositions.

### (a) CRISPR/Cas Systems

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary structures (e.g., hairpins) and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. Some Cas genes comprise homologous secondary and/or tertiary structures.

In some aspects, the CRISPR system used in the compositions herein comprises a Type II or a Type V CRISPR system.

### Type II CRISPR Systems

crRNA biogenesis in a Type II CRISPR system in nature requires a trans-activating CRISPR RNA (tracrRNA). The tracrRNA can be modified by endogenous RNaseIII, and then hybridizes to a crRNA repeat in the pre-crRNA array. Endogenous RNaseIII can be recruited to cleave the pre-crRNA. Cleaved crRNAs can be subjected to exoribonuclease trimming to produce the mature crRNA form (e.g., 5′ trimming). The tracrRNA can remain hybridized to the crRNA, and the tracrRNA and the crRNA associate with a site-directed polypeptide (e.g., Cas9). The crRNA of the crRNA-tracrRNA-Cas9 complex can guide the complex to a target nucleic acid to which the crRNA can hybridize. Hybridization of the crRNA to the target nucleic acid can activate Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096): 816-821 (2012) showed that the CRISPR/Cas9 system is useful for RNA-programmable genome editing, and international patent application publication number WO2013/176772 provides numerous examples and applications of the CRISPR/Cas endonuclease system for site-specific gene editing.

### Type V CRISPR Systems

Type V CRISPR systems have several important differences from Type II systems. For example, Cpf1 is a single RNA-guided endonuclease that, in contrast to Type II systems, lacks tracrRNA. In fact, Cpf1-associated CRISPR arrays can be processed into mature crRNAs without the requirement of an additional trans-activating tracrRNA. The Type V CRISPR array can be processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems can start with 20-24 nucleotides of spacer sequence followed by about 22 nucleotides of direct repeat. Also, Cpf1 can utilize a T-rich protospacer-adjacent motif (PAM) such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

In various aspects, the composition may comprise one or more RNA-guided endonucleases and one or more gRNAs. Exemplary endonucleases and gRNAs that may be used in the compositions herein are described below.

### Exemplary Endonucleases

Accordingly, in various aspects, the compositions may comprise one or more RNA-guided endonucleases (e.g., CRISPR-associated endonucleases). In various aspects, the RNA-guided endonuclease may comprise a Cas12a/Cpf1 endonuclease or functional variant thereof. The Cas12a/Cpf1 endonuclease (referenced herein interchangeably as Cas12a, Cpf1, or Cas12a/Cpf1) has a PAM sequence of TTTN, where N is any nucleotide. In various aspects, the compositions herein comprise a Cpf1 endonuclease or a nucleic acid encoding the Cpf1 endonuclease. In some aspects, the Cpf1 endonuclease is a Acidaminococcus sp. Cpf1. In some aspects, the Cpf1 endonuclease is enhanced or altered to expand its target range as described in Kleinstiver, B. P., Sousa, A. A., Walton, R. T., Tak, Y. E., Hsu, J. Y., Clement, K., Welch, M. M., Horng, J. E., Malagon-Lopez, J., Scarfo, I., et al. (2019). Engineered CRISPR-Cas12a variants with increased activities and improved targeting ranges for gene, epigenetic and base editing. Nat Biotechnol 37, 276-282, incorporated herein by reference in its entirety.

In other aspects, a suitable CRISPR associated endonuclease may comprise a Cas9 endonuclease or variant thereof. Suitable Cas9 endonucleases may preferably be an Staphylococcus aureus Cas9 (e.g., an saCas9) or any Cas9 endonuclease comprising a PAM sequence located near the intronic enhancer of the Klf1 gene, as described above. In various aspects, the compositions herein comprise an saCas9 endonuclease or a nucleic acid encoding the saCas9 endonuclease.

Genome-Targeting Nucleic Acid (gRNA)

The present disclosure provides a genome-targeting nucleic acid that can direct the activities of an associated polypeptide (e.g., an RNA-guided endonuclease) to a specific target sequence within a target nucleic acid. The genome-targeting nucleic acid can be an RNA. A genome-targeting RNA is referred to as a “guide RNA” or “gRNA” herein. A guide RNA can comprise at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex can bind a site-directed polypeptide, such that the guide RNA and RNA-guided endonuclease form a complex. The genome-targeting nucleic acid can provide target specificity to the complex by virtue of its association with the RNA-guided endonuclease. The genome-targeting nucleic acid thus can direct the activity of the RNA-guided endonuclease.

A double-molecule guide RNA can comprise two strands of RNA, which a single-molecule guide RNA (sgRNA) comprises a single strand of RNA. In a Type V system, the sgRNA can comprise, in the 5′ to 3′ direction, a minimum CRISPR repeat sequence and a spacer sequence.

By way of illustration, guide RNAs used in the CRISPR/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacer Sequence

The spacer sequence hybridizes to a sequence in a target nucleic acid of interest. The spacer of a genome-targeting nucleic acid can interact with a target nucleic acid in a sequence-specific manner via hybridization (i.e., base pairing). The nucleotide sequence of the spacer can vary depending on the sequence of the target nucleic acid of interest.

In a CRISPR/Cas system herein, the spacer sequence can be designed to hybridize to a target nucleic acid that is located 3′ of a PAM of the Cas12 enzyme used in the system. The spacer may perfectly match the target sequence or may have mismatches. Each Cas12 enzyme has a particular PAM sequence that it recognizes in a target DNA. For example, Cpf1 recognizes in a target nucleic acid a PAM that comprises the sequence 5′-TTTN-3′, where N is any nucleotide and N is immediately 5′ of the target nucleic acid sequence targeted by the spacer sequence.

The target nucleic acid sequence can comprise 20 nucleotides. The target nucleic acid can comprise less than 20 nucleotides. The target nucleic acid can comprise more than 20 nucleotides. The target nucleic acid can comprise at least: 5, 10, 15, 16, 17, 18, 19, 20, 21, 22, 23, 24, 25, 30 or more nucleotides. The target nucleic acid can comprise at most: 5, 10, 15, 16, 17, 18, 19, 20, 21, 22, 23, 24, 25, 30 or more nucleotides. The target nucleic acid sequence can comprise 23 bases immediately 3′ of the last nucleotide of the PAM. In one aspect, the PAM is suitable for a Cpf1 endonuclease, as described above. Suitable PAMs are also known in the art for other Cas nucleases (e.g., S. aureus Cas9) that may be used in this invention. The target nucleic acid sequence is often referred to as the PAM strand, and the complementary nucleic acid sequence is often referred to the non-PAM strand. One of skill in the art would recognize that the spacer sequence hybridizes to the non-PAM strand of the target nucleic acid. One of skill in the art would also recognize that the target nucleic acid and the gRNA spacer sequence may be understood to comprise the same sequence (or a sequence having close homology—as discussed below) wherein the target nucleic acid comprises DNA nucleotides and the gRNA spacer sequence comprises RNA nucleotides. In other words, a gRNA may be understood to “correspond to” a given DNA target sequence and may be immediately derived or contemplated from a given DNA target sequence.

The spacer sequence that hybridizes to the target nucleic acid can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer sequence can comprise 19 nucleotides. In some examples, the spacer sequence can comprise 18 nucleotides. In some examples, the spacer sequence can comprise 22 nucleotides. In some examples, the spacer sequence can comprise 23 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which may be thought of as a bulge or bulges.

The spacer sequence can be designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

In various aspects, the gRNAs useful in the present disclosure may comprise a spacer sequence corresponding to tgtccctggagctggggggggag (SEQ ID NO: 2) or gggaagtgggacagacagacagg (SEQ ID NO: 3). Variants of these gRNAs are also envisioned (e.g., gRNAs comprising a spacer sequence corresponding to a target sequence having at least 80%, at least 85%, at least 90%, at leat 95%, at least 96%, at least 97%, at least 98%, at least 99% sequence identify to SEQ ID NO: 2 or 3). Also provided are gRNAs comprising a spacer sequence comprising SEQ ID NO: 8 or SEQ ID NO: 9. In various aspects, the gRNAs target sequences 3′ to and adjacent to a PAM having a nucleotide sequence of TTTN, where N is any nucleotide. For ease of reference, exemplary gRNA target sequences and spacer sequences are provided below along with their respective PAMs. Dashes are used to indicate the location of the PAM relative to the target sequence in the target gene locus.

| gRNA target sequence | SEQ ID NO: | PAM |
|---|---|---|
| -TGTCCCTGGAGCTGGGGGGGGAG | 2 | TTTC- |
| GGGAAGTGGGACAGACAGACAGG- | 3 | -CAAA |

| gRNA spacer sequence | SEQ ID NO: | PAM |
|---|---|---|
| -UGUCCCUGGAGCUGGGGGGGGAG | 8 | UUUC- |
| GGGAAGUGGGACAGACAGACAGG- | 9 | -CAAA |

In general, a guide polynucleotide can complex with a compatible nucleic acid-guided nuclease and can hybridize with a target sequence, thereby directing the nuclease to the target sequence. A subject nucleic acid-guided nuclease capable of complexing with a guide polynucleotide can be referred to as a nucleic acid-guided nuclease that is compatible with the guide polynucleotide. In addition, a guide polynucleotide capable of complexing with a nucleic acid-guided nuclease can be referred to as a guide polynucleotide or a guide nucleic acid that is compatible with the nucleic acid-guided nucleases.

In some embodiments, a gRNA herein can include modified or non-naturally occurring nucleotides. In some embodiments a gRNA can be encoded by a DNA sequence on a polynucleotide molecule such as a plasmid, linear construct, or editing cassette as disclosed herein.

### Ribonucleoproteins

In some embodiments, gRNAs and endonucleases described herein may be complexed as a ribonucleoprotein (RNP). RNPs promote stability and facilitate delivery of the CRISPR components described herein. In various aspects, each RNP comprises a single gRNA and a single endonuclease. Therefore, when a composition herein comprises more than one gRNA (e.g., two or more gRNAs), it also may comprise more than one RNP (e.g., two or more RNP). When two or more RNPs are provided in the compositions herein, each may comprise a endonuclease (e.g., Cpf1 or a Cas9 as described above) and a gRNA targeting a nucleic acid in an intron of the Klf1 gene.

Accordingly, in various aspects, the gene-editing compositions provided herein can include (1) a guide RNA molecule (gRNA) as disclosed herein comprising a spacer sequence (which is capable of hybridizing to the genomic DNA target sequence), and sequence which is capable of binding to a Cas, e.g., Cas12 enzyme, and (2) a Cas endonuclease (E.g., Cas12a or Cpf1 endonuclease). In some aspects, the engineered CRISPR gene editing system comprises a gRNA targeting a sequence of SEQ ID NO: 3 and a CRISPR associated endonuclease (e.g., Cas12a or Cpf1). In some aspects, the engineered CRISPR gene editing system comprises a first gRNA comprising a spacer sequence corresponding to SEQ ID NO: 2 a second gRNA comprising a spacer sequence corresponding to SEQ ID NO: 3, and a CRISPR associated endonuclease (e.g., Cas12a or Cpf1).

### (b) Transcription Activator-Like Effector Nucleases (TALENs)

In various aspects, the gene-editing compositions may comprise one or more Transcription Activator-Like Effector Nucleases (TALENs).

TALENs represent another format of modular nucleases whereby an engineered DNA binding domain is linked to the FokI nuclease domain, and a pair of TALENs operate in tandem to achieve targeted DNA cleavage. The TALEN DNA binding domain derives from TALE proteins, which were originally described in the plant bacterial pathogen Xanthomonas sp. TALEs are comprised of tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9 or CRISPR/Cpf1 “nickase” mutants in which one of the Cas9 or Cpf1 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science, 2009 326(5959):1509-12; Mak et al., Science, 2012, 335(6069):716-9; and Moscou et al., Science, 2009, 326(5959):1501. The use of TALENs based on the “Golden Gate” platform, or cloning scheme, has been described by multiple groups; see, e.g., Cermak et al., Nucleic Acids Res., 2011, 39(12):e82; Li et al., Nucleic Acids Res., 2011, 39(14):6315-25; Weber et al., PLOS One., 2011, 6(2):e16765; Wang et al., J Genet Genomics, 2014, 41(6):339-47.; and Cermak T et al., Methods Mol Biol., 2015 1239:133-59.

In various aspects, the compositions provided herein comprise one or more TALENs. In some aspects, the compositions comprise two TALENs. In some aspects, the one or more TALENs target nucleic acids sequences at or near the Klf1 intronic enhancer (e.g., target sequences between nucleotides 149 to nucleotide 1059, between nucleotides 800 to 1000, between nucleotides 850 and 1000, or any sequence between 10, 20, 30, 40 or 50 basepairs upstream or downstream of nucleotide 944 of SEQ ID NO: 1. For example, one TALEN may target a nucleic acid sequence 5′ upstream of the Klf1 intronic enhancer and the other TALEN may target a nucleic acid sequence 3′ downstream of the Klf1 intronic enhancer. In various aspects, each TALEN comprises at a Target-Specific TALE array (i.e., that targets the above referenced nucleic acids). In some aspects, Target-Specific TALE array comprise an amino acid sequence according to SEQ ID NO: 4 or 5. As noted above, each TALEN may further comprise a Fok1 variant. Each TALEN may further comprise optional components (e.g., a nuclear localization signal and/or a TAL N terminal domain). Suitable NLS signals are known in the art and may include MVYPYDVPDYAELPPKKKRKV (SEQ ID NO: 10). Suitable TAL N terminal domains that may be used include DLRTLGYSQQQQEKIKPKVRSTVAQHHEALVGHGFTHAHIVALSQHPAALGTVAVKYQDMI AALPEATHEAIVGVGKQWSGARALEALLTVAGELRGPPLQLDTGQLLKIAKRGGVTAVEAV HAWRNALTGAPL (SEQ ID NO: 11). Suitable Fok1 variants that may be used include

(SEQ ID NO: 12)
IVAQLSRPDPALAALLVKSELEEKKSELRHKLKYVPHEYIELIEIARNP
TQDRILEMKVMEFFMKVYGYRGEHLGGSRKPDGAIYTVGSPIDYGVIVD
TKAYSGGYNLPIGQADAMQSYVEENQTRNKHINPNEWWKVYPSSVTEFK
FLFVSGHFKGNYKAQLTRLNHITNCNGAVLSVEELLIGGEMIKAGTLTL
EEVRRKFNNGEINFLD.

In some aspects, the one or more TALENs comprise an amino acid sequence comprising SEQ ID NO: 4 or 5. In various aspects, a composition provided herein comprises a first TALEN having an amino acid sequence comprising SEQ ID NO: 4 and a second TALEN having an amino acid sequence comprising SEQ ID NO: 5. In some aspects, the one or more TALENs comprise an amino acid sequence comprising SEQ ID NO: 6 or 7. In various aspects, a composition provided herein comprises a first TALEN having an amino acid sequence comprising SEQ ID NO: 6 and a second TALEN having an amino acid sequence comprising SEQ ID NO: 7. For ease of reference, amino acid sequences of illustrative full TALENs (SEQ ID NOs: 6 or 7) that may be used in the disclosed compositions are provided below, where the target specific TALE array (SEQ ID NOs: 4 or 5) is bolded, the NLS (SEQ ID NO: 10) is bolded and underlined, the N-terminal domain is italicized, and the heterodimer specific Fok1 variant (SEQ ID NO: 12) is underlined.

| FULL TALEN AMINO ACID SEQUENCE | SEQ ID NO |
|---|---|
| MVYPYDVPDYAELPPKKKRKVGIRIQDLRTLGYSQQQQEKIKPKVRST | 6 |
| VAQHHEALVGHGFTHAHIVALSQHPAALGTVAVKYQDMIAALPEATHEA | |
| IVGVGKQWSGARALEALLTVAGELRGPPLQLDTGQLLKIAKRGGVTAV | |
| EAVHAWRNALTGAPLNLTPAQVVAIASNGGGKQALETVQRLLPVLCQ | |
| DHGLTPAQVVAIASNIGGKQALETVQRLLPVLCQAHGLTPDQVVAIAS | |
| NNGGKQALETVQRLLPVLCQAHGLTPAQVVAIASNIGGKQALETVQR | |
| LLPVLCQAHGLTPAQVVAIASNNGGKQALETVQRLLPVLCQDHGLTP | |
| AQVVAIASNNGGKQALETVQRLLPVLCQAHGLTPEQVVAIASNNGGK | |
| QALETVQRLLPVLCQAHGLTPAQVVAIASNNGGKQALETVQRLLPVL | |
| CQDHGLTPDQVVAIASHDGGKQALETVQRLLPVLCQAHGLTPAQVVA | |
| IASNIGGKQALETVQRLLPVLCQAHGLTPEQVVAIASNNGGKQALETV | |
| QRLLPVLCQAHGLTPDQVVAIASNGGGKQALETVQRLLPVLCQDHGL | |
| TPAQVVAIASNNGGKQALETVQRLLPVLCQDHGLTPEQVVAIASNGG | |
| GKQALETVQRLLPVLCQAHGLTPAQVVAIASNGGGKQALETVQRLLP | |
| VLCQDHGLTPDQVVAIASNNGGKQALETVQRLLPVLCQAHGLTPEQV | |
| VAIASNNGGKQALESIVAQLSRPDPALAALLVKSELEEKKSELRHKLKY | |
| VPHEYIELIEIARNPTQDRILEMKVMEFFMKVYGYRGEHLGGSRKPDGA | |
| IYTVGSPIDYGVIVDTKAYSGGYNLPIGQADAMQSYVEENQTRNKHINP | |
| NEWWKVYPSSVTEFKFLFVSGHFKGNYKAQLTRLNHITNCNGAVLSVE | |
| ELLIGGEMIKAGTLTLEEVRRKFNNGEINFLD | |
| MVYPYDVPDYAELPPKKKRKVGIRIQDLRTLGYSQQQQEKIKPKVRST | 7 |
| VAQHHEALVGHGFTHAHIVALSQHPAALGTVAVKYQDMIAALPEATHEA | |
| IVGVGKQWSGARALEALLTVAGELRGPPLQLDTGQLLKIAKRGGVTAV | |
| EAVHAWRNALTGAPLNLTPAQVVAIASHDGGKQALETVQRLLPVLCQ | |
| AHGLTPDQVVAIASNGGGKQALETVQRLLPVLCQAHGLTPAQVVAIA | |
| SNGGGKQALETVQRLLPVLCQDHGLTPAQVVAIASNNGGKQALETV | |
| QRLLPVLCQDHGLTPEQVVAIASNGGGKQALETVQRLLPVLCQAHGL | |
| TPAQVVAIASNGGGKQALETVQRLLPVLCQDHGLTPAQVVAIASNGG | |
| GKQALETVQRLLPVLCQAHGLTPDQVVAIASNNGGKQALETVQRLLP | |
| VLCQAHGLTPAQVVAIASHDGGKQALETVQRLLPVLCQAHGLTPAQV | |
| VAIASHDGGKQALETVQRLLPVLCQAHGLTPAQVVAIASNGGGKQAL | |
| ETVQRLLPVLCQDHGLTPDQVVAIASNNGGKQALETVQRLLPVLCQD | |
| HGLTPEQVVAIASNGGGKQALETVQRLLPVLCQAHGLTPDQVVAIAS | |
| HDGGKQALETVQRLLPVLCQDHGLTPDQVVAIASNGGGKQALETVQ | |
| RLLPVLCQAHGLTPDQVVAIASNNGGKQALETVQRLLPVLCQAHGLT | |
| PDQVVAIASNGGGKQALETVQRLLPVLCQAHGLTPEQVVAIASHDGG | |
| KQALESIVAQLSRPDPALAALLVKSELEEKKSELRHKLKYVPHEYIELIEI | |
| ARNPTQDRILEMKVMEFFMKVYGYRGEHLGGSRKPDGAIYTVGSPIDY | |
| GVIVDTKAYSGGYNLPIGQADAMQSYVEENQTRNKHINPNEWWKVYP | |
| SSVTEFKFLFVSGHFKGNYKAQLTRLNHITNCNGAVLSVEELLIGGEMIK | |
| AGTLTLEEVRRKFNNGEINFLD* | |

### Zinc Finger Nucleases

In various aspects, the gene editing compositions herein may comprise a zinc finger nuclease. Zinc finger nucleases (ZFNs) are modular proteins comprised of an engineered zinc finger DNA binding domain linked to the catalytic domain of the type II endonuclease FokI. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZFN is typically comprised of 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers. In most applications of ZFNs, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these FokI variants.

A variety of ZFN-based systems have been described in the art, modifications thereof are regularly reported, and numerous references describe rules and parameters that are used to guide the design of ZFNs; see, e.g., Segal et al., Proc Natl Acad Sci, 1999 96(6):2758-63; Dreier B et al., J Mol Biol., 2000, 303(4):489-502; Liu Q et al., J Biol Chem., 2002, 277(6):3850-6; Dreier et al., J Biol Chem., 2005, 280(42):35588-97; and Dreier et al., J Biol Chem. 2001, 276(31):29466-78.

### (d) Isolated Nucleic Acids and Vectors

In various aspects, one or more components of the gene editing system provided herein (e.g., the gRNA, the CRISPR-associated endonuclease, and/or any other nuclease) may be encoded by a nucleic acid (e.g., those described above). Accordingly, provided herein are isolated nucleic acids encoding one or more gRNAs described above. Also provided are isolated nucleic acids encoding a CRISPR associated endonuclease. Other aspects provide for isolated nucleic acids encoding one or more of the TALE nuclease described above.

Polynucleotide sequences encoding a component of the gene editing systems described herein can include one or more vectors. The term “vector” as used herein can refer to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Other vectors (e.g., non-episomal mammalian vectors) can be integrated into the genome of a host cell upon introduction into the host cell. Recombinant expression vectors can include a nucleic acid of the present inventive concept in a form suitable for expression of the nucleic acid in a host cell, can mean that the recombinant expression vectors include one or more regulatory elements, which can be selected on the basis of the host cells to be used for expression, that is operatively-linked to the nucleic acid sequence to be expressed.

In some embodiments, a regulatory element can be operably linked to one or more elements of a targetable gene editing system herein so as to drive expression of the one or more components of the targetable gene editing system.

In some embodiments, a vector can include a regulatory element operably linked to a polynucleotide sequence encoding a nuclease (e.g., a Cas12, a Cas9, or a TALEN) described herein. The polynucleotide sequence encoding the nuclease herein can be codon optimized for expression in particular cells, such as prokaryotic or eukaryotic cells. Eukaryotic cells can be yeast, fungi, algae, plant, animal, or human cells. Eukaryotic cells can be those derived from a particular organism, such as a mammal, including but not limited to human, mouse, rat, rabbit, dog, or non-human mammal including non-human primate. Plant cells can include, without limitation, cells from seeds, suspension cultures, embryos, meristematic regions, callus tissue, leaves, roots, shoots, gametophytes, sporophytes, pollen and microspores.

As used herein, ‘codon optimization’ can refer to a process of modifying a nucleic acid sequence for enhanced expression in the host cells of interest by replacing at least one codon or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.”

In some embodiments, the components of the gene editing compositions (e.g., a CRISPR-associated endonuclease and one or more gRNAs in one instance, or one or more TALENs (or Zinc finger nuclease) in another) can be delivered either as DNA or RNA. When the gene editing composition comprises a CRISPR-associated endonuclease and one or more gRNA, both the endonuclease and guide nucleic acid may be delivered as RNA (unmodified or containing base or backbone modifications) so as to reduce the amount of time that the nucleic acid-guided nuclease persist in the cell (e.g. reduced half-life). This can reduce the level of off-target cleavage activity in the target cell. Since delivery of a CRISPR associated endonuclease as mRNA takes time to be translated into protein, an aspect herein can include delivering a guide nucleic acid several hours following the delivery of the endonuclease mRNA, to maximize the level of guide nucleic acid available for interaction with the nucleic acid-guided nuclease protein. In other cases, the CRISPR associated mRNA and guide nucleic acid can be delivered concomitantly. In other examples, the guide nucleic acid can be delivered sequentially, such as 0.5, 1, 2, 3, 4, or more hours after the CRISPR associated mRNA

In some embodiments, guide nucleic acid (e.g., gRNA) in the form of RNA or encoded on a DNA expression cassette can be introduced into a host cell that includes a nucleic acid-guided nuclease encoded on a vector or chromosome. The guide nucleic acid can be provided in the cassette having one or more polynucleotides, which can be contiguous or non-contiguous in the cassette. In some embodiments, the guide nucleic acid can be provided in the cassette as a single contiguous polynucleotide. In other embodiments, a tracking agent can be added to the guide nucleic acid in order to track distribution and activity.

In other embodiments, a variety of delivery systems can be used to introduce a gRNA and/or Cas9 nuclease into a host cell. In accordance with these embodiments, systems of use for embodiments disclosed herein can include, but are not limited to, yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, and/or exosomes.

In some embodiments, methods are provided for delivering one or more polynucleotides, such as or one or more vectors or linear polynucleotides as described herein, one or more transcripts thereof, and/or one or proteins transcribed therefrom, to a host cell. In some embodiments, a CRISPR-associated endonuclease in combination with (and optionally complexed with) a guide nucleic acid is delivered to a cell.

In certain embodiments, conventional viral and non-viral based gene transfer methods can be used to introduce nucleic acids in cells, such as prokaryotic cells, eukaryotic cells, plant cells, mammalian cells, or target tissues. Such methods can be used to administer nucleic acids encoding components of a gene editing system herein to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Any gene therapy method known in the art is contemplated of use herein. Methods of non-viral delivery of nucleic acids include are contemplated herein. Adeno-associated virus (“AAV”) vectors can also be used to transduce cells with target nucleic acids, e.g., in the in vitro production of nucleic acids and peptides, and for in vivo and ex vivo gene therapy procedures.

In some embodiments, a nucleic acid encoding any of the gene editing components herein (e.g., gRNA, CRISPR-associated endonuclease and/or TALENs) can be delivered to a cell using an adeno-associated virus (AAV). AAVs are small viruses which integrate site-specifically into the host genome and can therefore deliver a transgene. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, any mammalian AAV serotypes can be used herein for delivering the encoding nucleic acids described herein. Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

In some embodiments, polynucleotides disclosed herein can be delivered to a cell using at least one AAV vector. An AAV vector typically comprises a protein-based capsid, and a nucleic acid encapsidated by the capsid. The nucleic acid may be, for example, a vector genome comprising a transgene flanked by inverted terminal repeats. The AAV “capsid” is a near-spherical protein shell that comprises individual “capsid proteins” or “subunits.” AAV capsids typically comprise about 60 capsid protein subunits, associated and arranged with T=1 icosahedral symmetry. When an AAV vector is described herein as comprising an AAV capsid protein, it will be understood that the AAV vector comprises a capsid, wherein the capsid comprises one or more AAV capsid proteins (i.e., subunits). Also described herein are “viral-like particles” or “virus-like particles,” which refers to a capsid that does not comprise any vector genome or nucleic acid comprising a transgene. The virus vectors of the present disclosure can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619. The virus vectors of the present disclosure can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the present inventive concept. Further, the viral capsid or genomic elements can contain other modifications, including insertions, deletions and/or substitutions.

In some embodiments, the isolated nucleic acids encoding a gRNA and/or the fusion proteins herein may be packaged into an AAV vector (e.g., a AAV-Cas9 vector). In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11 or any combination thereof.

Exemplary AAV vectors contain two ITR (inverted terminal repeat) sequences which flank a central sequence region comprising the CRISPR associated (Cas) nuclease or TALEN sequence. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV vector may encode for one or more nuclear localization signals (NLS). In some embodiments, the AAV vector contains 1, 2, 3, 4, or 5 nuclear localization signals. In some aspects, the nuclear localization sequence comprises MVYPYDVPDYAELPPKKKRKV (SEQ ID NO: ______). Other suitable NLS are known in the art.

In some embodiments, the AAV vector may comprise additional elements to facilitate packaging of the vector and expression of the endonuclease and/or gRNA. In some embodiments, the AAV vector may comprise a polyA sequence.

In some embodiments, the AAV may contain one or more promoters. In some embodiments, the one or more promoters drive expression of the gene editing components in a cell or tissue of interest. In some embodiments, the one or more promoters are blood cell progenitor promoters. Exemplary blood cell progenitor promoters include those from the erythropoietin receptor (EPOR) or the GATA1 transcription factor. In some embodiments, the one or more promoters are specific hematopoetic stem cells. Exemplary hematopoetic stem cell specific promoters include those from transcription factors SCL or RUNX1.

In some embodiments, the AAV vector may be optimized for production in yeast, bacteria, insect cells, or mammalian cells. In some embodiments, the AAV vector may be optimized for expression in human cells. In some embodiments, the AAV vector may be optimized for expression in a bacculovirus expression system.

In some embodiments, AAV vectors disclosed herein may be packaged into virus particles which can be used to deliver the genome for transgene expression in target cells. In some embodiments, AAV vectors disclosed herein can be packaged into particles by transient transfection, use of producer cell lines, combining viral features into Ad-AAV hybrids, use of herpesvirus systems, or production in insect cells using baculoviruses.

In some embodiments, methods of generating a packaging cell herein involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

In some embodiments, a host cell is transiently or non-transiently transfected with one or more vectors, linear polynucleotides, polypeptides, nucleic acid-protein complexes, or any combination thereof as described herein. In some embodiments, a cell can be transfected in vitro, in culture, or ex vivo. In some embodiments, a cell can be transfected as it naturally occurs in a subject. In some embodiments, a cell that is transfected can be taken from a subject. In some embodiments, the cell is derived from cells taken from a subject, such as a cell line.

In some embodiments, a cell transfected with one or more vectors, linear polynucleotides, polypeptides, nucleic acid-protein complexes, or any combination thereof as described herein may be used to establish a new cell line can include one or more transfection-derived sequences. In some embodiments, a cell transiently transfected with the components of an engineered nucleic acid-guided nuclease system as described herein (such as by transient transfection of one or more vectors, or transfection with RNA), and modified through the activity of an engineered nuclease complex, may be used to establish a new cell line can include cells containing the modification but lacking any other exogenous sequence

Other delivery systems for targeting the gene editing compositions herein to a cell are contemplated. For example, suitable gene editing compositions may comprise liposomes, nanoparticles, a vector or other suitable delivery system known in the art. Various pharmaceutical compositions that may be used to deliver the gene editing compositions in vivo are described below.

### (e). Pharmaceutical Compositions

Any of the gene editing compositions described above (comprising, for example, any of the gene editing components, encoding nucleic acids and/or viral vectors) may be formulated into a pharmaceutical composition. In some embodiments, pharmaceutical composition may further include one or more pharmaceutically acceptable carriers, diluents or excipients. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some embodiments, the pharmaceutical compositions or formulations can be for administration by intraosseous, subcutaneous, intramuscular, intravenous, or intraperitoneal injection. In some embodiments, the pharmaceutical compositions or formulations are for parenteral administration, such as intravenous, subcutaneous, intraosseous injection, or a combination thereof. In some embodiments, the pharmaceutical compositions or formulations are for direct injection into bone marrow. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating AAV particles in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

### III. METHODS

Further aspects of the present disclosure are directed to methods of using any of the gene editing compositions provided above to reduce expression of KLF1 in a cell. Other aspects are directed to methods of using the gene editing compositions provided herein to treat a β-hemoglobinopathy in a subject. Still other aspects are directed to methods of increasing expression of a gamma globinin a cell.

In one aspect, a method is provided for increasing expression of a gamma globinin a cell, the method comprising delivering to the cell any of the gene editing compositions provided herein wherein the gene editing compositions target an intron of a Klf1 gene locus to effect a deletion in the intron, thereby decreasing expression of KLF1 and increasing expression of the gamma globin in the cell. Any of the gene editing compositions described above may be used herein. For example, the gene editing composition may comprise one or more transcription activator-like effector nucleases (TALENs). In some instances, the one or more TALENs may each independently comprise an amino sequence of SEQ ID NO: 4 or 5. For example, two TALENs may be used, wherein one TALEN has an amino acid sequence of SEQ ID NO: 4 and the other has an amino acid sequence of SEQ ID NO: 5. As another example, the gene editing composition may comprise a CRISPR-based gene editing system. For example, the gene editing composition can comprise one or more gRNA and an RNA guided endonuclease. In some aspects, the RNA guided endonuclease comprises Cpf1. In other aspects, the one or more gRNAs may comprise a spacer sequence corresponding to SEQ ID NO: 2 or SEQ ID NO: 3. In some aspects, the one or more gRNAs may comprise a spacer sequence corresponding to SEQ ID NO: 3. In some aspects, the composition comprises two gRNAs wherein a first gRNA comprises a spacer sequence corresponding to SEQ ID NO: 2 and the second gRNA comprises a spacer sequence corresponding to SEQ ID NO: 3. In any of the gene editing compositions used in the methods herein, the one or more gRNA may be complexed with the RNA guided endonuclease to form a ribonucleoprotein. In various aspects, the method may comprise delivering to the cell a nucleic acid encoding a gRNA and/or endonuclease and/or TALEN described herein. The nucleic acid may be delivered in a viral vector. In various aspects, the cell may be in vivo, in vitro, or ex vivo.

In another aspect of the present disclosure, a method of treating a β-hemoglobinopathy in a subject in need thereof is provided, the method comprising: administering to the subject a therapeutic amount of a gene editing composition targeting an intron of a Klf1 gene in at least one cell in the subject to effect a deletion in intron 1, thereby reducing expression of KLF1, wherein reducing expression of KLF1 increases expression of the gamma-globin in the cell and alleviates the β-hemoglobinapathy in the subject.

In various aspects, the methods provide for deleting a portion of an intron of the Klf1 gene in a cell. As described above, the portion of the intron that is targeted for deletion comprises an intronic enhancer that has been surprisingly discovered to be required to sustain normal expression of KLF1. When this intronic enhancer is deleted, KLF1 expression decreases. As KLF1 is a major repressor of gamma globinexpression, a decrease in KLF1 leads to an increase in gamma globinexpression. And this, in turn, helps alleviate symptoms of β-hemoglobinopathies in a subject, a disease characterized by loss of β-globin (the primary globin expressed in adult and postnatal tissue). The methods described herein provide a surprising method of titrating expression of KLF1 to increase β-globin expression, without losing KLF1 entirely.

In various aspects, expression of KLF1 is reduced by about 30-80%, about 35 to 80%, about 40 to 80%, about 45 to 80%, about 30 to 75%, about 30 to 70%, about 30 to 65%, about 30 to 60%, about 30 to 55%, about 35 to 75%, about 40 to 70%, about 40 to 60%, or about 45 to 55%. In some aspects, expression of KLF1 is reduced by about 30%, 35%, 40%, 45%, 50%, 55%, 60%, 65%, 70%, 75%, or 80%. In some aspects, expression of KLF1 is reduce by about 50%. KLF1 expression may be measured according to any standard techniques in the art (i.e., using reporter assays, western blotting or protein quantification methods).

In various aspects, the deletion in the intron 1 of the Klf1 gene occurs between nucleotide 800 and 1000, between nucleotides 850 and 960, or between nucleotides 860 and 960 according to SEQ ID NO: 1. In various aspects, the deletion in the intron 1 of the Klf1 gene occurs between nucleotides 866 and 952 according to SEQ ID NO: 1. In various aspects, the deletion in the intron 1 of the Klf1 gene occurs between nucleotides 891 and 944 according to SEQ ID NO: 1. In various aspects, the deletion is about 25 to 50 base pairs in length. In various aspects, the deletion is about 25 to 45, about 25 to 40, about 25 to 35, or about 25 to 30 base pairs in length. In various aspects, the deletion is about 30 to 50, about 35 to 50, about 40 to 50, or about 45 to 50 base pairs in length. In various aspects, the deletion is about 30 to 45 or about 35 to 40 base pairs in length.

In various aspects, the gene editing composition comprises a CRISPR-Cas system (e.g., a Type II or Type V CRISPR system described above). In various aspects, the gene editing composition comprises one or more gRNAs delivered alongside a Cas12a/Cpf1 endonuclease or a vector encoding them. In some aspects, the gene editing composition comprises two gRNAs and a Cas12a/Cpf1 endonuclease or a nucleic acid encoding it. In some aspects, the gene editing composition comprises one gRNA and a Cas12a/Cpf1 endonuclease or encoding nucleic acid. In various aspects the gRNA comprise a spacer sequence corresponding to SEQ ID NO: 3. In other aspects, the two or more gRNAs comprise spacer sequences corresponding to SEQ ID NO: 2 and SEQ ID NO: 3. Other CRISPR-Cas systems are contemplated, including CRISPR-Cas9 systems and CRISPR systems using a Cas variant that may act as a repressor.

In further aspects, the gene editing composition may comprise a TALEN or Zinc Finger nuclease. In some aspects, the gene editing composition may comprise two or more TALENs or Zinc Finger nucleases. In various embodiments, the gene editing composition comprises one or more transcription activator-like effector nucleases (TALENs) targeting the intron of the Klf1 gene. In some aspects, the one or more TALENs comprise an amino acid sequence selected from any one of SEQ ID NOs 4 and 5. In various aspects, the method comprises delivering two TALENs (i.e., a first TALEN and a second TALEN) wherein the first TALEN comprises an amino acid sequence comprising SEQ ID NO: 4 and the second TALEN comprises an amino acid sequence comprising SEQ ID NO: 5.

In various aspects, the gene editing composition comprises a nanoparticle, a liposome, a lentivirus, an adenovirus or other suitable delivery medium as described above.

In various aspects, the gene editing composition is intended to alter KLF1 expression in red blood cell progenitors. Red blood cells are differentiated from multipotent hematopoietic stem cells, isolated via their CD34 cell surface antigen from a suitable source (typically bone marrow). These can be cultured ex vivo under specific conditions that direct differentiation towards downstream lineages, including erythroid cells. Alternatively, they can be reintroduced into a recipient and differentiation will proceed in vivo. As a result, targeting the CD34+ cell enables expansion and generation of suitably edited cells that can be analyzed ex vivo or in vivo for the desired effect. Accordingly, in various aspects, the gene editing composition is administered as a suitable pharmaceutical formulation to the bone marrow or blood progenitor cell pool in a subject. For example, in some instances, the gene editing composition may be administered via intraosseous infusion. In still other aspects, the gene editing composition may be administered directly into a bone marrow of a subject.

In various aspects, the methods of treating a β-hemoglobinapathy in a subject in need thereof further comprises administering a stem cell mobilizing agent. Suitable stem cell mobilizing agents include G-CSF or Plerixafor. In various aspects, the method provided herein comprises administering the gene editing composition intravenously into a subject after intravenous injection of the stem cell mobilizing agent like G-CSF or Plerixafor. In various aspects, the method provided herein comprises administering the gene editing composition subcutaneously into a subject after intravenous injection of the stem cell mobilizing agent like G-CSF or Plerixafor.

In various aspects, the methods comprise injecting the gene editing composition serially into a subject until a satisfactory level of gamma globin expression is achieved. In some aspects, a satisfactory level of gamma globin expression is at least about 20%, at least about 25%, at least about 30%, at least about 35%, or at least about 40% of total hemoglobin expression in the patient. For example, a satisfactory level of gamma globin expression may be at least 20% of total hemoglobin expression in the patient. Another satisfactory level of gamma globin expression may be at least 25% of total hemoglobin expression in the patient. Still another satisfactory level of gamma globin expression may be at least 30% of total hemoglobin expression in the patient. Another satisfactory level of gamma globin expression may be at least 35% of total hemoglobin expression in the patient. Still another satisfactory level of gamma globin expression may be at least 40% of total hemoglobin expression in the patient. Levels of gamma globin may be measured using standard methods in the art (e.g., using hemoglobin electrophoresis or hemoglobin fractionation as described in Mayo Protocols (e.g., Test HBEL1)).

Traditional methods for treating β-hemoglobinopathy usually comprise a form of myeloablative toxicity (e.g., using radiation or high dose chemotherapy). This allows for a stem cell transplant and replenishment of normal beta-globin producing cells. Advantageously, the methods provided herein do not require forms of myeloablative toxicity. Accordingly, treating the β-hemoglobinapathy using the methods herein does not comprise or result in myeloablative toxicity.

As used herein “treating” β-hemoglobinopathy according to methods of the present disclosure may comprise an improvement in anemia, reduction in hemolysis, reduction of organ injury, relief from pain, decreased frequency of acute and chronic complications, improved physical function, and/or an improved quality of life.

Suitable β-hemoglobinapathies that may be treated according to the methods and compositions of the instant disclosure include sickle cell anemia and beta-thalassemia.

A suitable subject herein includes a human, a livestock animal, a companion animal, a lab animal, or a zoological animal. In some embodiments, the subject may be a rodent, e.g., a mouse, a rat, a guinea pig, etc. In some embodiments, the subject may be a livestock animal. Non-limiting examples of suitable livestock animals may include pigs, cows, horses, goats, sheep, llamas and alpacas. In some embodiments, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In various embodiments, compositions disclosed herein may be administered by parenteral administration. As used herein, “by parenteral administration” refers to administration of the compositions disclosed herein via a route other than through the digestive tract. In some embodiments, compositions disclosed herein may be administered by parenteral injection. In some aspects, administration of the disclosed compositions by parenteral injection may be by subcutaneous, intramuscular, intravenous, intraperitoneal, or intraosseous injection. In some embodiments, administration of the disclosed compositions by parenteral injection may be by slow or bolus methods as known in the field. In some embodiments, administration of the disclosed compositions may be by serial injection. In some embodiments, the route of administration by parenteral injection can be determined by the target location. In some embodiments, compositions disclosed herein may be formulated for parenteral administration by intraosseous injection. In some embodiments, compositions disclosed herein may be formulated for parenteral administration by direct injection to a bone marrow. In some embodiments, compositions disclosed herein may formulated for parenteral administration by intravenous injection.

In various embodiments, the dose of compositions disclosed herein to be administered are not particularly limited and may be appropriately chosen depending on conditions such as a purpose of preventive and/or therapeutic treatment, a type of a disease, the body weight or age of a subject, severity of a disease and the like. In some embodiments, administration of a dose of a composition disclosed herein may comprise a therapeutically effective amount of the composition disclosed herein. As used herein, the term “therapeutically effective” refers to an amount of administered composition that treats β-hemoglobinopathy, reduces presentation of at least one symptom associated with β-hemoglobinopathy, reduces anemia, reduces hemolysis, reduces organ injury, relieves pain, decreases frequency of acute and/or chronic conditions associated with β-hemoglobinopathy, improves physical function, improves quality of life, increases survivability, or a combination thereof.

In some embodiments, a composition disclosed herein may be administered to a subject in need thereof once. In some embodiments, a composition disclosed herein may be administered to a subject in need thereof more than once. In some embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

The number of times a composition may be administered to a subject in need thereof can depend on the discretion of a medical professional, the severity of the heart disease, and the subject's response to the formulation. In some embodiments, a composition disclosed herein may be administered continuously; alternatively, the dose of composition being administered may be temporarily reduced or temporarily suspended for a certain length of time (i.e., a “composition holiday”). In some aspects, the length of the composition holiday can vary between 2 days and 1 year, including by way of example only, 2 days, 1 week, 1 month, 6 months, and 1 year. In another aspect, dose reduction during a composition holiday may be from 10%-100%, including by way of example only 10%, 25%, 50%, 75%, and 100%.

In various embodiments, the desired daily dose of compositions disclosed herein may be presented in a single dose or as divided doses administered simultaneously (or over a short period of time) or at appropriate intervals. In other embodiments, administration of a composition disclosed herein may be administered to a subject about once a day, about twice a day, about three times a day. In still other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of a composition disclosed herein may be administered to a subject once about 4 weeks.

In some embodiments, a composition as disclosed may be initially administered followed by a subsequent administration of one for more different compositions or treatment regimens. In other embodiments, a composition as disclosed may be administered after administration of one for more different compositions or treatment regimens.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1—Identification of a Mutation at the Intronic Enhancer Region in a JMML Patient with Increased HbF

In order to investigate KLF1 gene status in cells from patients that exhibit high fetal hemoglobin protein (HbF), the complete Klf1 transcription unit was sequenced from genomic DNA from 20 juvenile myelomonocytic leukemia (JMML) patient samples. JMML is an early childhood myeloproliferative/myelodysplastic disease and clonal disorder of pluripotent stem cells. JMML patients present with thrombocytopenia and hepatosplenomegaly as well as abnormally high HbF levels that correlate with disease progression.

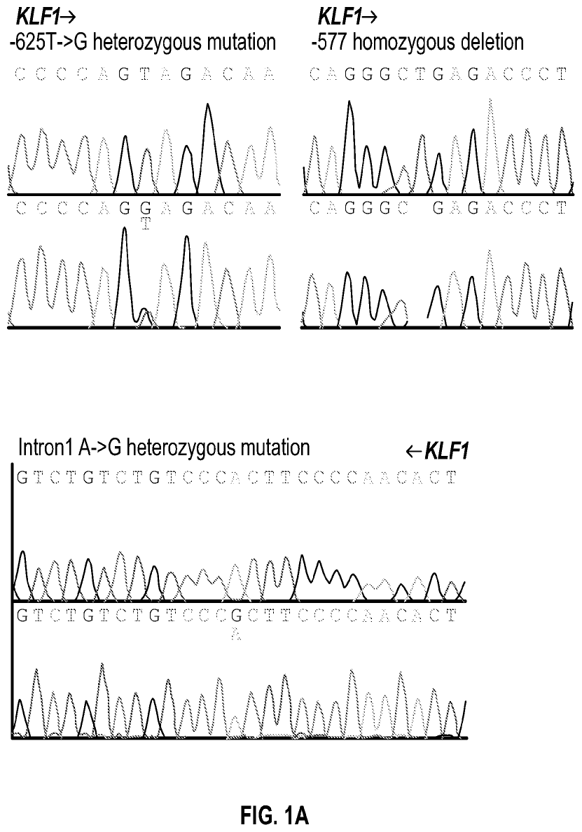

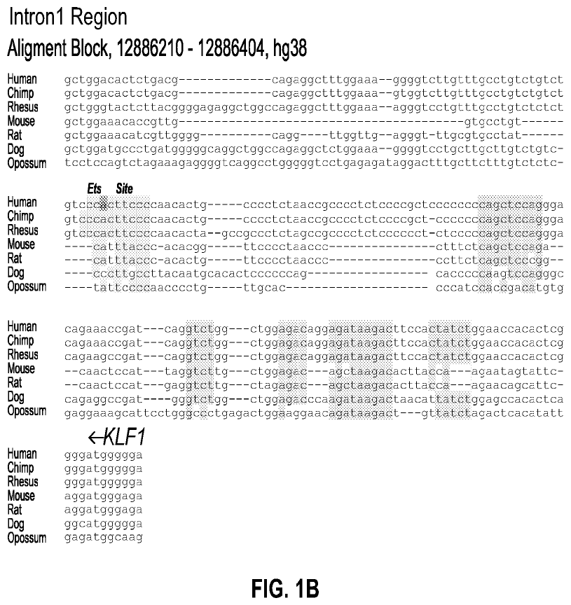

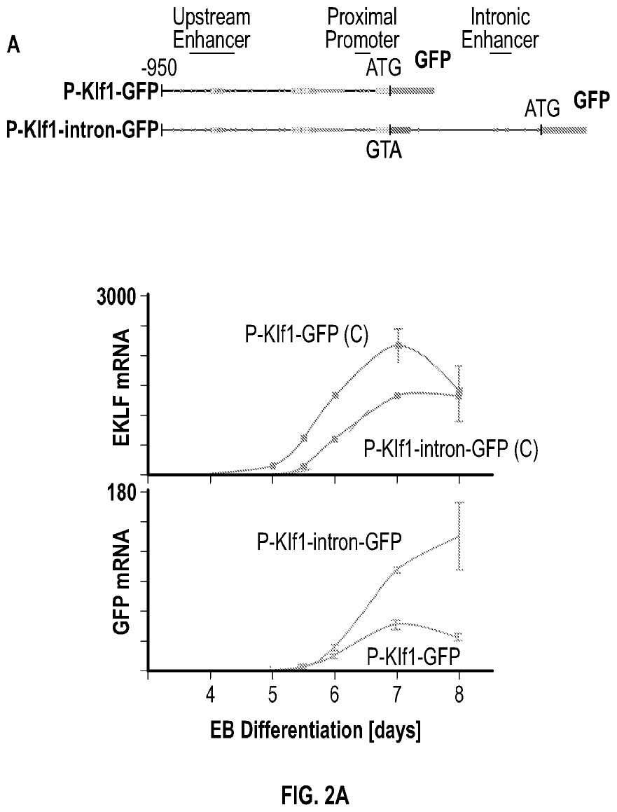

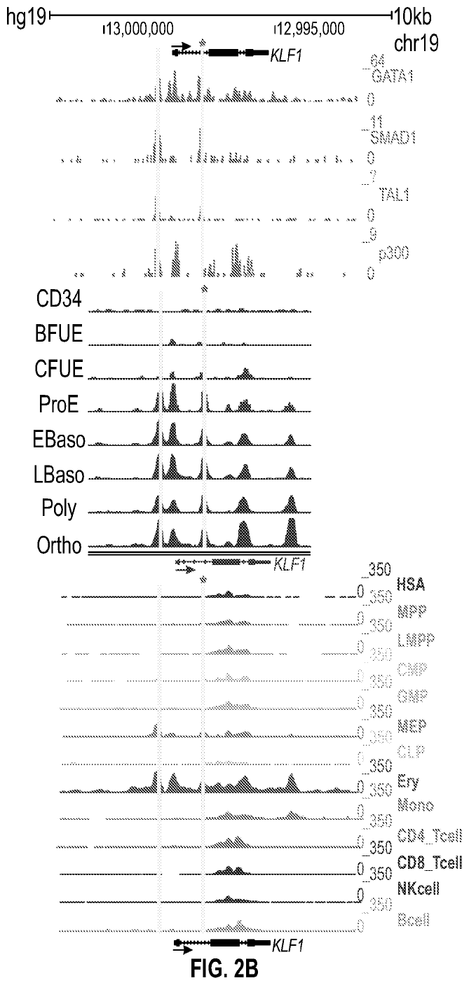

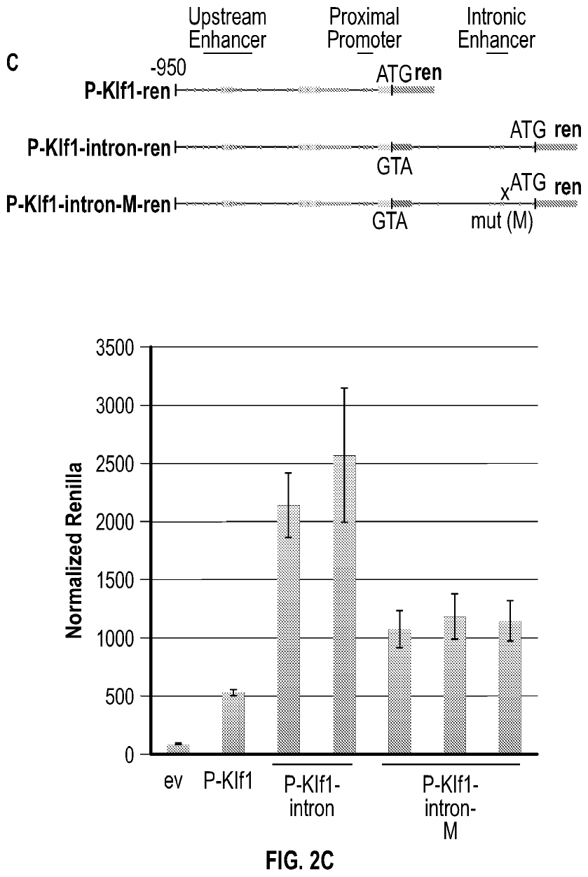

Analysis of the Klf1 transcription unit from patient samples identified three changes within the samples, two located in the upstream promoter (Table 1, FIG. 1A). Of particular interest, one sample from a patient with a slightly higher than normal HbF (HM3554) contains a single heterozygous base mutation within a highly conserved region of intron 1 that is not a SNP (Table 1, FIG. 1B). This change did not appear in analysis of over 5000 genomes from direct sequencing and from public databases. Further, the mutation is located near an intronic enhancer region which leads to a 30-fold increase in linked reporter activity beyond that seen within the 5′-promoter alone, as assayed in differentiating mouse embryoid bodies derived from mouse embryonic stem cells (FIG. 2A).

TABLE 1
JMML patient samples

| | UPN | sample ID | Source* | Hb F % | comments |
|---|---|---|---|---|---|
| 1 | 987 | HM1432 | BM | | |
| 2 | 1358 | HM2109 | PB | 42 | Homozygous −577 |
| | | | | | del, rs201870270, |
| | | | | | MAF = 0.8% |
| 3 | 1441 | HM2176 | BM | | |
| 4 | 1484 | HM2230 | PB | | |
| 5 | 1603 | HM2429 | PB | | |
| 6 | 1669 | HM2534 | PB | | |
| 7 | 1716 | HM2620 | BM | | |
| 8 | 1905 | HM2936 | PB | | |
| 9 | 1911 | HM2948 | BM | 9.7 | |
| 10 | 1965 | HM3049 | BM | 19.7 | |
| 11 | 2024 | HM3152 | BM | | |
| 12 | 2048 | HM3200 | PB | | |
| 13 | 2065 | HM3235 | PB | | |
| 14 | 2099 | HM3294 | PB | 3.4 | Heterozygous −625 |
| | | | | | T > G mutation; non- |
| | | | | | conserved region |
| 15 | 2104 | HM3304 | PB | 57.1 | |
| 16 | 2114 | HM3314 | BM | 6.9 | |
| 17 | 2218 | HM3554 | PB | 4 | Heterozygous T > C, |
| | | | | | non-SNP mutation; |
| | | | | | intron 1 conserved |
| | | | | | region |
| 18 | 2292 | HM3662 | BM | 26 | |
| 19 | 2301 | HM3679 | BM | 20 | |
| 20 | 2309 | HM3690 | BM | 4.6 | |

*BM = bone marrow, PB = peripheral blood

Closer analysis revealed that the mutation is located within a sequence that is highly conserved across mammalian species, and directly adjacent to previously identified GATA and SMAD recognition elements in the mouse (FIG. 1B). This region binds GATA1, SMAD1 and TAL1 transcription factors also in human erythroid cells (FIG. 2B), and the increase in chromatin accessibility during human hematopoiesis and particularly during erythroid differentiation as judged by ATAC seq analysis is centered at that site (FIG. 2B). These data suggest that the intron 1 site mutated in the JMML patient is within a functionally important element that is critical for optimal mammalian KLF1 expression.

### Example 2—Report Assays Demonstrate the Relevance of the Intronic Enhancer Mutation on KLF1 Expression

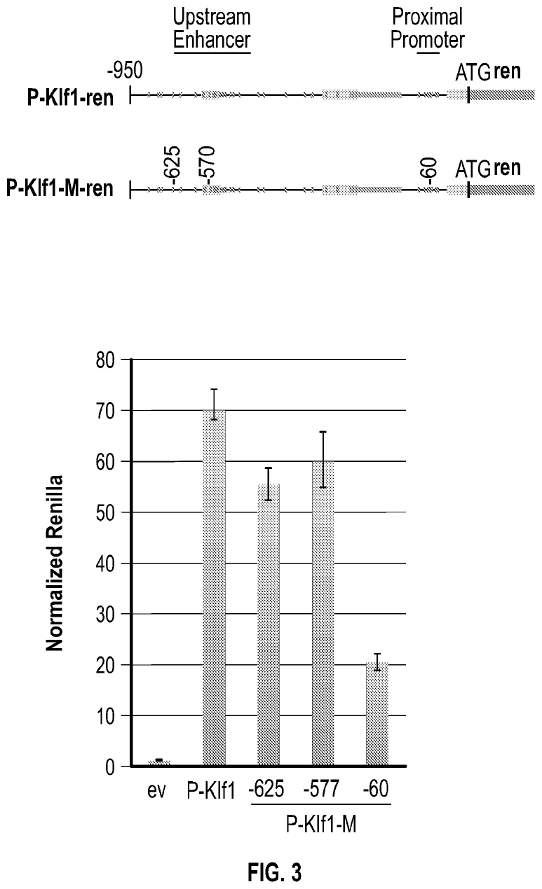

Reporter assays were performed in transfected JK1 human erythroleukemia cell lines to address the importance of the intron 1 mutation. Specifically, cells were transfected with of renilla reporters driven by wild-type or site-directed mutated KLF1 promoter and intronic regions. FIG. 2C shows that inclusion of intron 1 increases the activity of the promoter ˜4-fold, but this increase is reduced by 50% when the same assay is performed using a reporter with an intron 1 single nucleotide mutation. In contrast, performing the same assay with two upstream promoter changes had little effect (FIG. 3).

### Example 3—Genome Editing at the Mutated Intronic Region in the KU812 Human Erythroleukemia Cell Line Leads to Reduced KLF1 and Elevated Epsilon Globin Expression

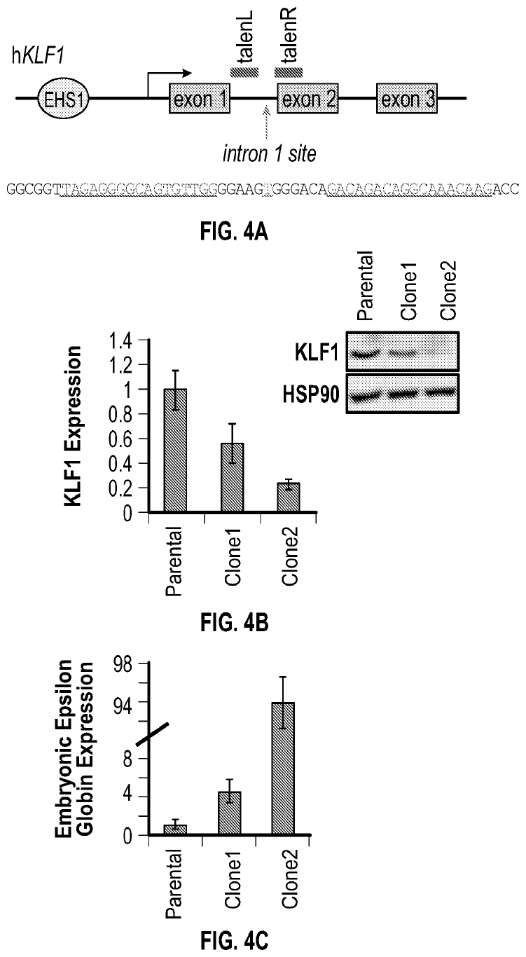

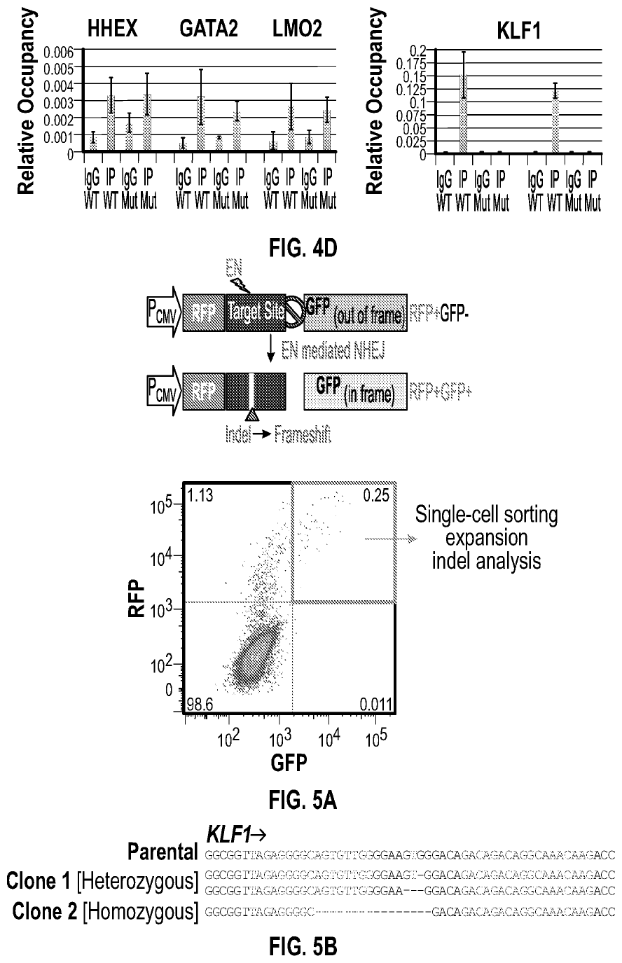

To determine whether the sequence surrounding the mutation identified in Examples 1 and 2 is critical for hKLF1 transcription, TALEN directed nuclease technology (as generally described in Joung, J. Keith, and Jeffry D. Sander. “TALENs: a widely applicable technology for targeted genome editing.” Nature reviews Molecular cell biology 14.1 (2013): 49-55; and Joung and Sander, 2013; and Miller, J. C., Tan, S., Qiao, G., Barlow, K. A., Wang, J., Xia, D. F., Meng, X., Paschon, D. E., Leung, E., Hinkley, S. J., et al. (2011). A TALE nuclease architecture for efficient genome editing. Nat Biotechnol 29, 143-148, each incorporated herein by reference in their entirety) was used to delete the site within the KLF1 genome in human cells (FIG. 4A), and KLF1 levels were monitored. Selection of cells bearing the deletion was aided by using a cotransfected RFP/GFP reporter (FIG. 5A). Using this system, human KU812 cells were transfected and double positive RFP+/GFP+ cells were selected for analysis (FIG. 5A).

The clones encompassed homo-and heterozygous deletions of 3 to 15 nt centered at the intron 1 mutation site (e.g., FIG. 5B), indicating successful attainment of genomic editing. Clones of non-TALEN treated parental cells were also obtained to serve as a control to avoid any variables from clonal selection. Western and qRT-PCR analysis was performed for clone 1 (heterozygous deletion), clone 2 (homozygous deletion), and one of the parental clones (FIG. 5B). Western analysis shows that KLF1 protein levels are quantitatively reduced in clone 1 and more reduced in clone 2 compared to a parental clone, effects that mirror the KLF1 transcript expression (FIG. 4B). Globin expression analysis reveals that while β- and γ-globin expression were unaffected (not shown), ε-globin levels are dramatically upregulated in the KU812 cell line in proportion to KLF1 expression (FIG. 4C), consistent with the dysregulation of ε-globin seen in human KLF1 mutant patients with chronic nonspherocytic hemolytic anemia.

Although γ-globin levels were not affected in this cell line (as they are already quite high to begin with in the parental cell (not shown)), these results sub-localized the functionally critical sequence from within the original large 0.9 KB intron 1 to a small ˜15 bp region surrounding the intron 1 site mutation and suggest that a circumscribed deletion centered on this mutation site within normal cells will quantitatively decrease, but not ablate, KLF1 expression.

### Example 4—Identification of TEL/ETV6 Protein Interaction with the KLF1 Intron 1 Region

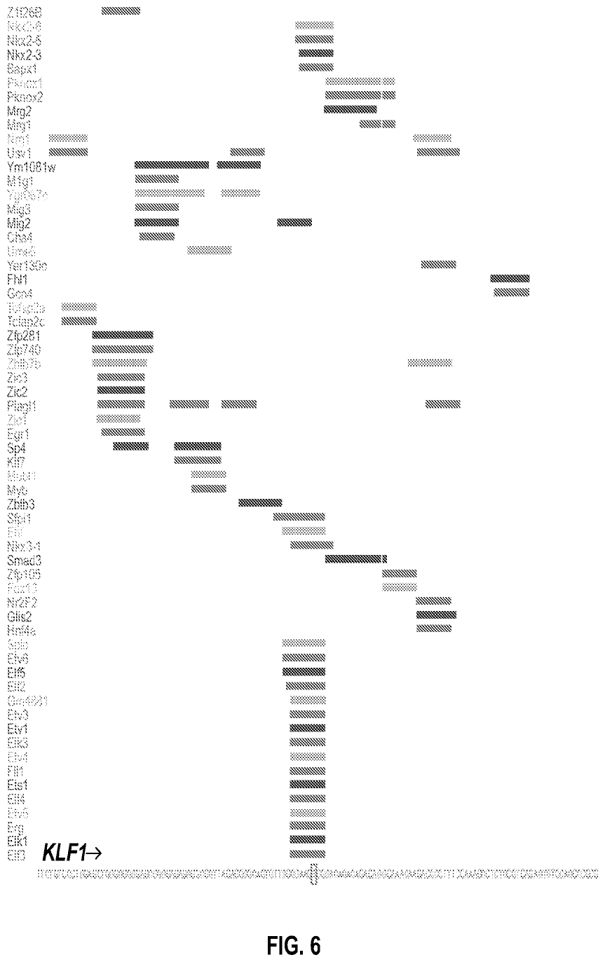

Given the high level of sequence conservation at this site (FIG. 1B), transcription factor interactions may be predicted to be negatively affected by the mutation. To test this, the Universal PBM Resource for Oligonucleotide Binding Evaluation (UniPROBE) factor interaction prediction analysis (described in Hume, Maxwell A., et al. “UniPROBE, update 2015: new tools and content for the online database of protein—binding microarray data on protein—DNA interactions.” Nucleic acids research 43.D1 (2015): D117-D122; and Newburger, Daniel E., and Martha L. Bulyk. “UniPROBE: an online database of protein binding microarray data on protein—DNA interactions.” Nucleic acids research 37.suppl_1 (2009): D77-D82, each incorporated herein by reference in their entirety) was used to identify binding sites that are directly disrupted by the intronic mutation coupled to analysis via the NIH Roadmap Epigenomics Consortium (FIG. 6). This analysis showed that the intron region surrounding the mutated site is enriched for binding sites for the ETS family of proteins that interact with a consensus motif known to be important for the transition from stem to erythroid cells. The single base intron 1 mutation is predicted to disrupt the binding for these proteins. Exclusion of those transcription factors not expressed in erythroid cells (analysis via ErythronDB (Kingsley, Paul D., et al. “Ontogeny of erythroid gene expression.” Blood, The Journal of the American Society of Hematology 121.6 (2013): e5-e13) and BioGPS (Wu, Chunlei, et al. “BioGPS: building your own mash-up of gene annotations and expression profiles.” Nucleic acids research 44.D1 (2016): D313-D316)) leaves only a small subset as possible contenders for positive binding such as ERG, ETV6, and ELF2. Of particular interest is TEL/ETV6, a protein that, similar to KLF1, has known roles in enhancing erythroid differentiation and hemoglobin synthesis and in inhibiting megakaryopoiesis. In vivo ChIP analyses of WT and intron 1-edited KU812 cells show that TEL/ETV6 binds to this site, and that its binding is abrogated when the target site is mutated (FIG. 4D). This data suggests that the TEL/ETV6 protein joins GATA1/SMAD/TAL1 in forming an optimal protein/DNA complex at this critical site within intron 1 of the KLF1 gene.

### Example 5—Genome Editing of KLF1 Intron 1 in Human CD34+ Cells Using TALENs

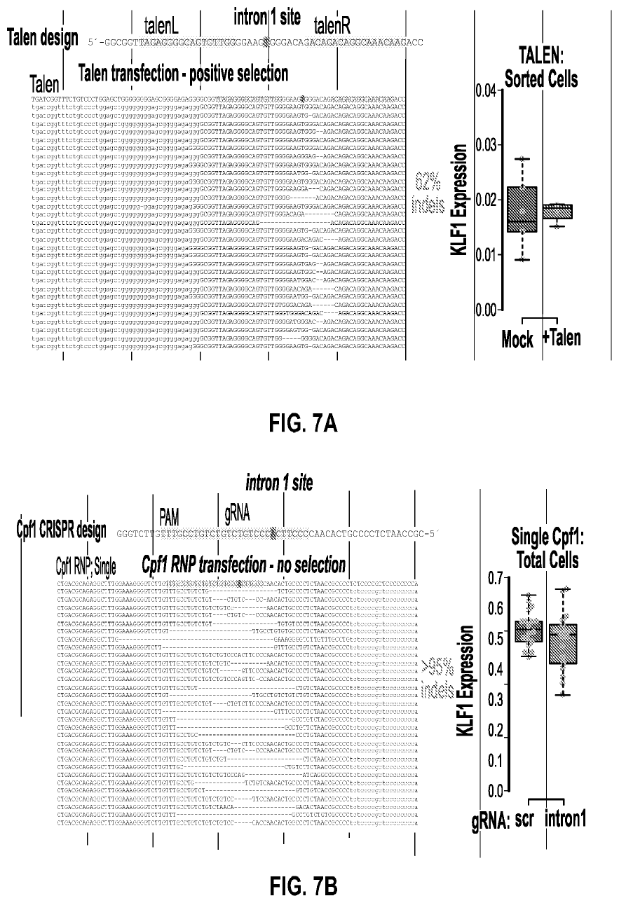

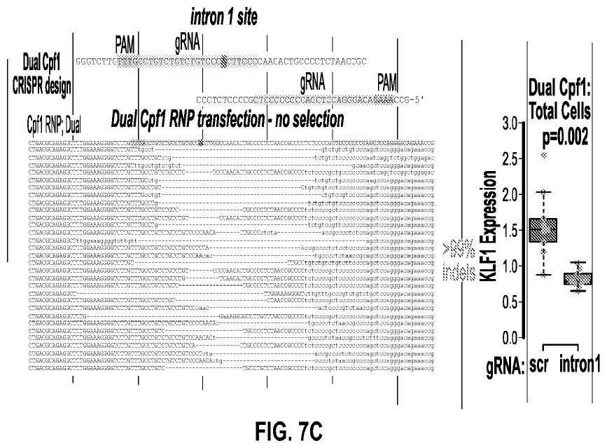

To test whether quantitative knock down of KLF1 expression could be attained by editing of intron 1 in primary cells, human CD34+ cells were expanded and transfected with the verified TALEN arms, followed by differentiation towards the erythroid lineage using a three-step protocol (Antoniani et al., 2018; Breda et al., 2016; Grevet et al., 2018; Wu et al., 2019). Positively-transfected cells were selected and were cultured as a pool of cells. Sorted cells from transfections without the TALEN arms served as the unaltered control. Deep sequencing of the positive pool indicates that over 60% of the cells are edited at the expected deletion site that overlaps the intron 1 mutation (FIG. 7A). Although encouraging, it was found that the resultant small deletions had little effect on KLF1 expression, possibly due to the fact that the efficiency was not near 100%, viability was not good after the DNA transfection, and at most a 30% drop in expression was expected (50% effect x ˜60% cells with deletion).

### Example 6—Genome Editing of KLF1 Intron 1 in Human CD34+ Cells Using CRISPR-Cas12a

In another experiment, a CRISPR-Cas12a system was used to modify KLF1 intron1 in human CD34+ cells. Cas12a (Cpf1) was chosen as the endonuclease because the PAM sequence required by Cas9 limited the target sequences to regions not near the site of interest in intron 1. Cpf1/Cas12a, in contrast, requires a significantly different PAM sequence (TTTV), and has been optimized (e.g., Acidaminococcus sp variant) for increased activity and fidelity (Kleinstiver, B. P., Sousa, A. A., Walton, R. T., Tak, Y. E., Hsu, J. Y., Clement, K., Welch, M. M., Horng, J. E., Malagon-Lopez, J., Scarfo, I., et al. (2019). Engineered CRISPR-Cas12a variants with increased activities and improved targeting ranges for gene, epigenetic and base editing. Nat Biotechnol 37, 276-282, incorporated herein by reference in its entirety). Accordingly, a Cpf1 gRNA was designed that directly overlaps the site of interest and exhibits no predicted off-targets even with up to 2 mismatches (CRISPOR program: Concordet, J. P. and Haeussler, M. (2018). CRISPOR: intuitive guide selection for CRISPR/Cas9 genome editing experiments and screens. Nucleic Acids Res 46, W242-W245). The Cpf1 gRNA was complexed with the Cpf1 endonuclease in a ribonucleoprotein (RNP) in order to allow delivery into CD34+ cells at high efficiency while retaining high cell viability. When this new design/approach was tested, it was found that even in the absence of selection, >95% of the cells are edited (FIG. 7B). In contrast, use of a scrambled Cpf1/gRNA control routinely gives close to zero editing. The deletions at the region of interest are larger than had been observed with TALENs, but surprisingly a significant effect on KLF1 levels was still not observed on average even though the range of effect was wide (i.e., a subset of the data points were decreased compared to the control).

It is possible that Cpf1-directed deletion using a single gRNA at the KLF1 intron 1 site might be too limited. Although one might predict that disruption of transcription factor binding should have a major effect on activity of enhancer function, it has been observed that removal of a single site in vivo can have a surprisingly minor effect, as any associated ‘enhanceosome’ components may still form if those protein-protein interactions are strong, and if there are other adjacent DNA binding sites that retain recruitment and cooperativity of other components in the full complex. Indeed, the KLF1 gene forms an extended 3D complex with multiple enhancers in its vicinity. To that end, the sequence surrounding the original Cpf1 position was searched, and an additional Cpf1 PAM-sequence target site was identified ˜35 bp away that also exhibits no predicted off-targets even with up to 2 mismatches (CRISPOR program). It was postulated that introduction of both Cpf1 gRNA sequences might enable a larger deletion region to be generated. This dual Cpf1 approach was tested. The results of this experiment showed a high efficiency of editing in the absence of selection (>99%), but importantly also created a larger deletion and produced the anticipated 50% drop in KLF1 levels (FIG. 7C). This suggests that focused disruption of the ETV6 site is not sufficient, but rather that deletion of a slightly larger region, perhaps containing other important binding sites, is needed to disrupt the enhancing activity of intron 1. Importantly, this verifies the importance of the intron 1 region for optimal KLF1 expression in vivo in primary cells and sets the stage for detailed analysis of its downstream effects.

### Example 7—Methods

KU812 and JK1 cells lines have been described (Drexler, H. G. (2010). Guide to Leukemia-Lymphoma Cell Lines (2nd edn). Braunschweig). Patient samples were as previously published (Stieglitz, E., Taylor-Weiner, A. N., Chang, T. Y., Gelston, L. C., Wang, Y. D., Mazor, T., Esquivel, E., Yu,A., Seepo, S., Olsen, S. R., et al. (2015). The genomic landscape of juvenile myelomonocytic leukemia. Nat Genet 47, 1326-1333). Genomic sequencing using primers spanning the complete KLF1 transcription unit, including the upstream promoter, was performed in both directions as previously described (Gnanapragasam, M. N., Crispino, J. D., Ali, A. M., Weinberg, R., Hoffman, R., Raza, A. and Bieker, J. J. (2018). Survey and evaluation of mutations in the human KLF1 transcription unit. Sci Rep 8, 6587).

Reporter assays were performed after cotransfection of renilla reporter constructs into JK1 cells using X-tremegene HP transfection reagent (sigma) along with a luciferase expressing plasmid for normalization (Siatecka, M. and Bieker, J. J. (2011). The multifunctional role of EKLF/KLF1 during erythropoiesis. Blood 118, 2044-2054). Assays were performed using the dual luciferase kit (Promega) according to manufacturer's instructions. Mutagenesis was performed with the Quick-change kit (Stratagene).

Mouse embryonic stem cell culture and differentiation into embryoid bodies was performed as described using cell lines generated by targeting the P-Klf1-GFP or PKlf1-intron-GFP constructs into the site-specific Ainv18 homing site (Lohmann, F. and Bieker, J. J. (2008). Activation of Eklf expression during hematopoiesis by Gata2 and Smad5 prior to erythroid commitment. Development 135, 2071-2082). These contain a single copy, unidirectionally inserted sequence into the same site and avoids random integration.

RNA isolation and RT-qPCR analysis was as previously described (Gnanapragasam et al., 2016; Lohmann and Bieker, 2008, described above).

CD34+ cells were purchased from AllCells or obtained from the Yale Cooperative Center of Excellence in Hematology. These were differentiated under three-phase protocols, initially based on (Antoniani, C., Meneghini, V., Lattanzi, A., Felix, T., Romano, O., Magrin, E., Weber, L., Pavani, G., El Hoss, S., Kurita, R., et al. (2018). Induction of fetal hemoglobin synthesis by CRISPR/Cas9-mediated editing of the human beta-globin locus. Blood 131, 1960-1973; and Yien, Y. Y. and Bieker, J. J. (2012). Functional interactions between erythroid Kruppel-like factor EKLF/KLF1) and protein phosphatase PPM1B/PP2Cbeta. J Biol Chem 287, 15193-15204 (2013). EKLF/KLF1, a tissue-restricted integrator of transcriptional control, chromatin remodeling, and lineage determination. Mol Cell Biol 33, 4-13) but then later based on (Breda, L., Motta, I., Lourenco, S., Gemmo, C., Deng, W., Rupon, J. W., Abdulmalik, O. Y., Manwani, D., Blobel, G. A. and Rivella, S. (2016). Forced chromatin looping raises fetal hemoglobin in adult sickle cells to higher levels than pharmacologic inducers. Blood 128, 1139-1143; Grevet, J. D., Lan, X., Hamagami, N., Edwards, C. R., Sankaranarayanan, L., Ji, X., Bhardwaj, S. K., Face, C. J., Posocco, D. F., Abdulmalik, O., et al. (2018). Domain-focused CRISPR screen identifies HRI as a fetal hemoglobin regulator in human erythroid cells. Science 361, 285-290; and Wu, Y., Zeng, J., Roscoe, B. P., Liu, P., Yao, Q., Lazzarotto, C. R., Clement, K., Cole, M. A., Luk, K., Baricordi, C., et al. (2019). Highly efficient therapeutic gene editing of human hematopoietic stem cells. Nat Med 25, 776-783). Transfection was performed after the expansion phase I, and cells were allowed to recover for 24-48 hrs in phase I media prior to switching to phase II.

Cloning of left and right TALEN arms was performed by insertion into a CMV promoter containing plasmid, downstream of T7 polymerase promoter, HA tag, and NLS sequences, but upstream (in frame) of the Fok1 nuclease and bGH poly (A) signals (PNA Bio Inc). In addition, an RFP/GFP surrogate reporter was synthesized that contained the intron 1 sequence between them as a linker, but out of frame for GFP. Proper nuclease activity after transfection/expression leads to frameshift mutations and expression of GFP, enabling enrichment of a low number of positively transfected cells by sorting for RFP+/GFP+ double positivity (FIG. 5A) (Kim, H., Um, E., Cho, S. R., Jung, C. and Kim, J. S. (2011). Surrogate reporters for enrichment of cells with nuclease-induced mutations. Nat Methods 8, 941-943 (Kim et al., 2011).

Transfection of TALEN DNAs into KU812 cells was performed using the Neon Transfection System (1450 v, 10 ms, 3 pulses), sorted for RFP+/GFP+, and the positive pools were diluted to single cells into 96-well plates. After expansion, DNA was isolated and analyzed by PCR at the region of interest (using sequencing primer pairs (Gnanapragasam et al., 2018, cited above)) to determine whether any of these clones contained a deletion. Individual clones that were positive were expanded and used for subsequent analyses. Genomic PCR of cell line material used 25 ng purified DNA (Qiagen), followed by direct sequence analysis of the product (Macrogen).

CD34+ cells were transfected with the Amaxa Nucleofector II using program U-008 for TALEN DNA. For RNP we used the Amaxa 4D Nucleofector with program EO-100. Biological replicates were each analyzed in triplicate.

AsCpf1 protein (enhanced as described in Kleinstiver, B. P., Sousa, A. A., Walton, R. T., Tak, Y. E., Hsu, J. Y., Clement, K., Welch, M. M., Horng, J. E., Malagon-Lopez, J., Scarfo, I., et al. (2019). Engineered CRISPR-Cas12a variants with increased activities and improved targeting ranges for gene, epigenetic and base editing. Nat Biotechnol 37, 276-282) was purchased from IDT. RNP were freshly formed by mixing 105 pmol of Cpf1 protein with 120 pmol of gRNA (IDT) for 15 at room temperature, and kept on ice until use.

Positive pools of CD34+ cells were analyzed by next generation sequencing (MGH DNA Core Facility) following genomic DNA isolation and PCR. Alternatively, TIDE (Brinkman, E. K., Chen, T., Amendola, M. and van Steensel, B. (2014). Easy quantitative assessment of genome editing by sequence trace decomposition. Nucleic Acids Res 42, e168) or ICE (Hsiau, T., Conant, D., Rossi, N., Maures, T., Waite, K., Yang, J., Joshi, S., Kelso, R., Holden, K., Enzmann, B., et al. (2019). Inference of CRISPR Edits from Sanger Trace Data. BioRxiv.) analysis was performed on these or on KU812 cell DNA samples using sequencing primer pairs (Gnanapragasam et al., 2018, cited above).

Predicted off-target effects of the TALEN pair was tested using the PROGNOS analysis program (Fine, E. J., Cradick, T. J., Zhao, C. L., Lin, Y. and Bao, G. (2014). An online bioinformatics tool predicts zinc finger and TALE nuclease off-target cleavage. Nucleic Acids Res 42, e42). No predicted off-targets were found when 0, or even 3, mismatches were allowed per arm (Table 2). Predicted off-targets for the two gRNA designs used the CRISPOR program (Concordet, J. P. and Haeussler, M. (2018). CRISPOR: intuitive guide selection for CRISPR/Cas9 genome editing experiments and screens. Nucleic Acids Res 46, W242-W245), which showed no predicted off-targets even allowing for up to 2 mismatches (Table 3, parameters: genome: hg38 and PAM: TTTV).

TABLE 2
PROGNOS Summary
Parameters
The hg19 genome was searched for the TALEN pair targeting:
5′-TAGAGGGGCAGTGTTGG NN...NN GACAGACAGGCAAACAAG-3′ (SEQ ID NO: 13 and
SEQ ID NO: 14)
Using these RVDs:
Left:
01NI02NN03NI04NN05NN06NN07NN08HD09NI10NN11NG12NN13NG14NG15NN16NN
Search 1: Up to 0 mismatches allowed in each nuclease half-site.
Allowed spacing distances were: 10 11 12 13 14 15 16 17 18 19 20 21 22 23 24 25 26 27
28 29 30 Homodimers and heterodimers of the nucleases were included in the output
1 total sites were located Mismatch Types Genomic Regions
0 and 0: 1
Exons: 0
Promoters: 0
Introns: 1
Intergenic: 0
The top 1 sites are ranked below according to parameters optimized for TALEN off-target
prediction.
Rank: 1 Homology Score: 2 TALEN Score: 100 Genomic Region: Intron Closest Gene:
KLF1
Search 2: Up to 3 mismatches allowed in each nuclease half-site.
Allowed spacing distances were: 10 11 12 13 14 15 16 17 18 19 20 21 22 23 24 25 26 27
28 29 30 Homodimers and heterodimers of the nucleases were included in the output
1 total sites were located Mismatch Types Genomic Regions
0 and 0: 1
Exons: 0
Promoters: 0
Introns: 1
Intergenic: 0
The top 1 sites are ranked below according to parameters optimized for TALEN off-target
prediction.
Rank: 1 Homology Score: 32 TALEN Score: 100 Genomic Region: Intron Closest Gene:
KLF1

TABLE 3
CRISPOR analysis of gRNA designs

| | | Off-targets for | offtargetCount | |
|---|---|---|---|---|
| | PAM- | 0-1-2-3-4 | targetGenome | DeepCpf1- |
| guideId | TargetSeq | mismatches | GeneLocus | Score |
| 28forw | TTTC- | 0-0-0-2-30 | 32 | 7.54 |
| | TGTCCCTGGAGCTGGGGGGG | | intron:KLF1 | |
| | G (SEQ ID NO:_) | | | |
| 110rev | TTTG- | 0-0-0-7-78 | 85 | 28.59 |
| | CCTGTCTGTCTGTCCCACTTC | | intron:KLF1 | |
| | (SEQ ID NO:_) | | | |

Chromatin immunoprecipitation of TEL/ETV6 was performed with a mix of antibodies (containing anti-TEL from Santa Cruz sc-1668335 and sc-8547 along with home-made rabbit polyclonal; generous gifts from Drs B Graves and K Clark (Hollenhorst, P. C., McIntosh, L. P. and Graves, B. J. (2011). Genomic and biochemical insights into the specificity of ETS transcription factors. Annu Rev Biochem 80, 437-471)) using standard conditions. Positive control targets were chosen based on Unnikrishnan, A., Guan, Y. F., Huang, Y., Beck, D., Thoms, J. A., Peirs, S., Knezevic, K., Ma, S., de Walle, I. V., de Jong, I., et al. (2016). A quantitative proteomics approach identifies ETV6 and IKZF1 as new regulators of an ERG-driven transcriptional network. Nucleic Acids Res 44, 10644-10661. Primer pairs for detection at KLF1 intron 1 are provided in Gnanapragasam, M. N., Crispino, J. D., Ali, A. M., Weinberg, R., Hoffman, R., Raza, A. and Bieker, J. J. (2018). Survey and evaluation of mutations in the human KLF1 transcription unit. Sci Rep 8, 6587, which is incorporated herein by reference in its entirety.

Mutating the intron 1 region in HUDEP2 cells (Kurita, R., Suda, N., Sudo, K., Miharada, K., Hiroyama, T., Miyoshi, H., Tani, K. and Nakamura, Y. (2013)) was considered. Establishment of immortalized human erythroid progenitor cell lines able to produce enucleated red blood cells. PLOS One 8, e59890), as they have been widely utilized for human erythroid studies. However, we obtained aberrant results with all our directed mutagenesis attempts at the KLF1 genomic region. Karyotype analysis revealed genomic instability along with trisomy 19 (not shown), which is the genomic location for KLF1. As a result, we switched to using JK1 and/or KU812 human leukemia cell lines for our initial set of gene editing analyses, as both of these also express KLF1 (unlike K562) and appear more stable Bieker, J. J. (1996). Isolation, genomic structure, and expression of human Erythroid Kruppel-like Factor (EKLF). DNA and Cell Biol. 15, 347-352 (2010). Putting a finger on the switch. Nat Genet 42, 733-734; Li, B., Ding, L., Yang, C., Kang, B., Liu, L., Story, M. D. and Pace, B. S. (2014). Characterization of transcription factor networks involved in umbilical cord blood CD34+ stem cells-derived erythropoiesis. PLOS One 9, e107133 (Bieker, 1996; Li et al., 2014).

1. A method of treating a β-hemoglobinopathy in a subject in need thereof, the method comprising: administering to the subject a therapeutic amount of a gene editing composition targeting an intron of a Klf1 gene in at least one cell in the subject to effect a deletion in intron 1 between nucleotides 891 and 944 according to SEQ ID NO: 1, thereby reducing expression of Klf1, wherein reducing expression of Klf1 increases expression of the gamma-globin in the cell and alleviates the β-hemoglobinapthy in the subject.

2. The method of claim 1, wherein expression of Klf1 is reduced by about 30-80%.

3. (canceled)

4. The method of claim 1, wherein the deletion of intron 1 is about 25 to 50 bp in size.

5. (canceled)

6. The method of claim 1, wherein the gene editing composition comprises: (a) an RNA-guided endonuclease and (b) one or more gRNA or sgRNA or (c) one or more nucleic acids encoding the RNA-guided endonuclease and/or the one or more gRNAs or sgRNAs targeting the intron of the Klf1 gene.

7. The method of claim 6, wherein the RNA-guided endonuclease comprises a Cpf1 endonuclease.

8. The method of claim 6, wherein each gRNAs or sgRNAs forms a ribonucleoprotein complex with the RNA-guided endonuclease.

9. The method of claim 6, wherein the one or more gRNAs or sgRNAs comprise a spacer sequence corresponding to SEQ ID NO: 2 and/or SEQ ID NO: 3.

10. (canceled)

11. (canceled)

12. The method of claim 1, wherein the gene editing composition comprises one or more transcription activator-like effector nucleases (TALENs) targeting the intron of the Klf1 gene.

13. The method of claim 12, wherein the one or more TALENs comprise an amino acid sequence comprising SEQ ID NOs: 4 and/or SEQ ID NO: 5.

14. (canceled)

15. The method of claim 1, wherein the gene editing composition comprises a nanoparticle, a liposome, lentivirus, and/or adenovirus.

16. (canceled)

17. The method of claim 1, wherein the cell is a hematopoietic stem cell or other blood progenitor cell.

18. The method of claim 1, wherein the gene editing composition is administered via intraosseous infusion.

19. The method of claim 1, wherein the gene editing composition is directly injected into bone marrow of the subject.

20. The method of claim 1, further comprising administering a stem cell mobilizing agent.

21. The method of claim 20, wherein the gene editing composition is injected either intravenously or subcutaneously into the subject after intravenous administration of the stem cell mobilizing agent.

22. (canceled)

23. The method of claim 20, wherein the stem cell mobilizing agent comprises G-CSF, Plerixafor, or a combination thereof

24. The method of claim 1, wherein the gene editing composition is injected serially into the subject until a satisfactory level of gamma expression is achieved.

25. The method of claim 1, wherein the β-hemoglobinopathy is sickle cell anemia or beta-thalassemia.

26. The method of claim 1, wherein treating the β-hemoglobinopathy does not comprise or result in myeloablative toxicity.

27. The method of claim 1, wherein treating the β-hemoglobinopathy comprises reducing anemia, reducing hemolysis, reducing or preventing organ injury, relieving pain, decreasing frequency of acute and/or chronic complications, improving physical function, improving quality of life or any combination thereof.

28-34. (canceled)

35. A method of increasing expression of a gamma-globin in a cell, the method comprising: delivering to the cell: one or more nucleic acids encoding transcription activator-like effector nucleases (TALENs) targeting an intron of a Klf1 gene locus to effect a deletion in the intron between nucleotides 891 and 944 according to SEQ ID NO: 1, thereby reducing expression of Klf1, wherein reducing expression of Klf1 increases expression of the gamma globin in the cell.

36. The method of claim 35, wherein each of the one or more TALENs comprise an amino acid sequence comprising SEQ ID NO: 4 and/or SEQ ID NO: 5.

37-39. (canceled)

40. The method of claim 35, wherein expression of Klf1 is reduced by about 30-80%.

41. (canceled)

42. The method of claim 35, wherein the deletion is about 20 to 30 bp in size.

43. (canceled)

44. The method of claim 35, wherein the cell is a hematopoietic stem cell or other blood progenitor cell.

45. The method of claim 35, wherein the cell is either in vitro, in vivo, or ex vivo.

46. (canceled)

47. (canceled)

48. A gene editing composition comprising (a) an RNA-guided endonuclease or nucleic acid encoding the RNA-guided endonuclease, (b) one or more gRNAs or sgRNAs targeting an intron of a Klf1 gene locus between nucleotides 891 and 944 according to SEQ ID NO: 1 or a nucleic acid encoding the one or more gRNAs or sgRNAs and (c) a pharmaceutically acceptable carrier or excipient.

49. The gene editing composition of claim 48, wherein the RNA-guided endonuclease comprises Cpf1.

50. The gene editing composition of claim 48, wherein the one or more gRNAs or sgRNAs comprise a spacer sequence corresponding to SEQ ID NO: 2 and/or SEQ ID NO: 3.

51. (canceled)

52. (canceled)

53. The gene editing composition of claim 48, wherein each gRNA or sgRNA forms a ribonucleoprotein complex with at least one RNA endonuclease and the gene editing composition comprises the ribonucleoprotein complex.

54. The gene editing composition of claim 48, comprising one or more a nucleic acid encoding the RNA-guided endonuclease and/or the one or more gRNAs or sgRNAs.

55. A gene editing composition comprising (a) one or more transcription activator-like effector nucleases (TALENs) targeting an intron of a Klf1 gene locus and (b) a pharmaceutically acceptable carrier or excipient.

56. The gene editing composition of claim 55, wherein at least one TALEN comprises an amino acid sequence comprising SEQ ID NO: 4 or SEQ ID NO: 5.

57. (canceled)

58. The gene editing composition of claim 48, wherein the carrier or excipient comprises a liposome, a nanoparticle, a viral vector or any combination thereof.

59. The gene editing composition of claim 58, wherein the gene editing composition is formulated for intraosseous infusion or bone marrow injection.
